(19) United States
(12) Patent Application Publication
(10) Pub. No.: US 2009/0156545 A1
(43) Pub. Date: Jun. 18, 2009

(54) Substituted Phosphate Esters of Nucleoside Phosphonates

(76) Inventors: Karl Y. Hostetler, Del Mar, CA (US); James R. Beadle, San Diego, CA (US); Jacqueline C. Ruiz, San Diego, CA (US)

Correspondence Address: JONES DAY, 222 EAST 41ST ST, NEW YORK, NY, 10017, US

(21) Appl. No.: 11/887,501

(22) PCT Filed: Mar. 30, 2006
(86) PCT No.: PCT/US2006/012121

§ 371 Date: Oct. 16, 2008

Related U.S. Application Data
(60) Provisional application No. 60/667,739, filed on Apr. 1, 2005.

(51) Int. Cl. A61K 31/675; C07H 19/20; C07H 19/10; C07F 9/6512; A61P 31/12; A61P 35/00

(52) U.S. Cl. 514/47; 514/49; 514/81; 514/86; 536/26.7; 536/26.8; 544/243; 544/244

(57) ABSTRACT

Compounds and compositions are provided for treatment, prevention, or amelioration of a variety of medical disorders associated with viral infections and/or cell proliferation. The compounds provided herein are obtained by attaching the phosphonate nucleoside of interest to alkyloxyalkyl-phosphate or alkyl-phosphate in a phosphate-phosphono anhydride linkage to provide a modified nucleoside phosphonate drug.

### RELATED APPLICATION DATA

This application claims priority under 35 U.S.C. §119(c) to U.S. provisional application Ser. No. 60/667,739, entitled “Substituted Phosphate Esters Of Nucleoside Phosphonates” to Hostetler et al., filed Apr. 1, 2005. The contents of the provisional application are incorporated by reference herein in their entirety.

### GRANT INFORMATION

This invention was made with government support under Grant No. AI29164 awarded by the National Institute of Allergy and Infectious Diseases/National Health Institute and Grant No. DAMD17-01-2-0071 awarded by United States Army. The United States government has certain rights in this invention.

### FIELD

Provided herein are antiviral and anticancer phosphonate drugs, their preparation, and their use for treatment of viral infections and cancers. Also provided are methods for synthesizing anhydrides containing alkyl phosphate or alkoxyalkyl phosphate coupled to nucleoside phosphonate drugs. The new conjugates have greater antiviral and/or antiproliferative activity when compared with the parent nucleoside phosphonate.

In another embodiment, provided herein are methods of treatment, prevention, or amelioration of a variety of medical disorders associated with viral infections and cell proliferation using the compounds and compositions provided herein.

### BACKGROUND

Phosphonate nucleosides are well known in the art and are in clinical use as antiviral and anticancer agents (see, Holy, A., Phosphonomethoxyalkyl analogs of nucleotides, Current Pharmaceutical Design 9(31), 2567-92, 2003). Their limitations relate to poor oral bioavailability, poor target cell uptake and toxicity in kidneys. In general, nucleoside phosphonate uptake into target cells is poor because of the dual negative charges on the phosphonate moiety. Once in the cell, they require two subsequent anabolic phosphorylations to achieve activity as the nucleoside phosphonate diphosphate. Some nucleoside phosphonates are hampered by slow phosphorylation.

There is a continuing need for less toxic, more effective pharmaceutical agents to treat a variety of disorders associated with viral infection, and cell proliferation.

### SUMMARY

Provided herein are nucleoside phosphonates linked via their phosphonate residue to the phosphate of alkoxyalkyl-phosphate, alkylglycerol-phosphate or alkyl-phosphate and pharmaceutically acceptable derivatives thereof. In certain embodiments, the nucleoside phosphonates or acyclic nucleoside phosphonates linked to the phosphate of alkoxyalkyl-phosphate, alkylglycerol-phosphate or alkyl-phosphate result in orally available compounds which exhibit greater antiviral or anticancer activity by promoting cell uptake and favorable cellular metabolism which yields the nucleoside phosphonate monophosphate, bypassing the need for the first of two anabolic phosphorylations. In certain embodiments, compounds provided herein exhibit greater antiviral or anticancer activity than the unmodified nucleoside phosphonates.

Also provided are compositions and methods of using the compounds and compositions for the treatment of various diseases. In one embodiment, compounds and compositions provided herein have antiviral activity. In another embodiment, provided herein are compounds and compositions that are useful in the treatment, prevention, or amelioration of one or more symptoms associated with cell proliferation.

In one embodiment, the compounds for use in the compositions and methods provided herein have formula I:

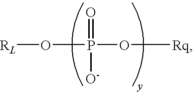

or pharmaceutically acceptable derivatives thereof,
wherein R_{L} is a lipophilic group, R_{q} is a pharmacologically active phosphonate or a phosphonate derivative of a pharmacologically active compound, coupled to the phosphate group by an anhydride linkage and y is 1 or 2.

Also provided are pharmaceutically-acceptable derivatives, including salts, esters, enol ethers, enol esters, solvates, hydrates and prodrugs of the compounds described herein. Further provided are pharmaceutical compositions containing the compounds provided herein and a pharmaceutically acceptable carrier. In one embodiment, the pharmaceutical compositions are formulated for single dosage administration.

Methods of treatment, prevention or amelioration using the compounds and compositions provided herein are provided. Such methods encompass treating, preventing or ameliorating one or more symptoms of diseases associated with viral infections and cell proliferation. In practicing the methods, effective amounts of the compounds or compositions containing therapeutically effective concentrations of the compounds are administered.

Articles of manufacture are provided containing packaging material, a compound or composition provided herein which is useful for treating, preventing, or ameliorating one or more symptoms of diseases or disorders associated with viral infections or cell proliferation using the compounds and compositions provided herein, and a label that indicates that the compound or composition is useful for treating, preventing, or ameliorating one or more symptoms of diseases or disorders associated with viral infections or cell proliferation.

### DETAILED DESCRIPTION

### A. Definitions

Unless defined otherwise, all technical and scientific terms used herein have the same meaning as is commonly understood by one of ordinary skill in the art to which the claimed subject matter belongs. All patents, applications, published applications and other publications are incorporated by reference in their entirety. In the event that there are a plurality of definitions for a term herein, those in this section prevail unless stated otherwise.

As used herein the terms “phosphonate” and “phosphonate group” mean a functional group or moiety within a molecule that comprises at least one phosphorus-carbon bond, and at least one phosphorus-oxygen double bond. The phosphorus atom is further substituted with oxygen, sulfur, and nitrogen substituents. These substituents may be part of a prodrug moiety. As used herein, “phosphonate” and “phosphonate group” include molecules with phosphonic acid, phosphonic monoester, phosphonic diester, phosphonamidate, phosphondiamidate, and phosphonthioate functional groups.

As used herein, the term “nucleoside” refers to a molecule composed of a heterocyclic base and a carbohydrate. A nucleoside is composed of a heterocyclic nitrogenous base in N-glycosidic linkage with a sugar. Nucleosides are recognized in the art to include natural bases (standard), and non-natural bases well known in the art. The carbohydrates include the true sugars found in natural nucleosides or a species replacing the ribofuranosyl moiety or acyclic sugars. The heterocyclic nitrogenous bases are generally located at the 1′ position of a nucleoside sugar moiety. Nucleosides generally contain a base and sugar group. The nucleosides can be unmodified or modified at the sugar, and/or base moiety, (also referred to interchangeably as nucleoside analogs, modified nucleosides, non-natural nucleosides, non-standard nucleosides; see for example, Eckstein et al., International PCT Publication No. WO 92/07065 and Usman et al., International PCT Publication No. WO 93/15187). In natural nucleosides the heterocyclic base is thymine, uracil, cytosine, adenine or guanine. In certain embodiments, acyclic sugars contain 3-6 carbon atoms and include, for example, the acyclic sugar moieties present in acyclovir (—CH₂—O—CH₂—CH₂—OH), ganciclovir (—CH₂—O—CH(CH₂OH)—CH₂—OH), and the like. Natural nucleosides have the β-D-configuration. The term “nucleoside” shall be understood to encompass unnatural configurations and species replacing the true sugar that lack an anomeric carbon. In natural nucleosides the heterocyclic base is attached to the carbohydrate is through a carbon-nitrogen bond. The term “nucleoside” shall be understood to encompass species wherein the heterocyclic base and carbohydrate are attached through a carbon-carbon bond (C-nucleosides).

Where the nucleoside contains 1 or more functional groups that may be reactive to form undesired products under the reaction conditions of the present process, for example, the amino groups of cytosine and adenine and the 2-amino and 6-oxo groups of guanine, such functional groups may be blocked using the protecting groups commonly employed in nucleoside chemistry. For example, the amino group of adenine and cytosine may be protected by benzoyl; the 6-oxo and 2-amino groups of guanine may be protected by the triphenylmethyl (trityl) group. The selection of methods for introducing and subsequent removal of such protecting groups are well known to one of ordinary skill in the pertinent art.

As used herein, the term “nucleoside base” refers to natural and non-natural purine and pyrimidine bases, including adenine, thymine, cytosine, guanine and uracil and analogs thereof.

The terms “nucleoside phosphonate” and “acyclic nucleoside phosphonate” refer to the group of phosphonomethoxyalkyl or phosphono substituted nucleoside derivatives that are biologically active, for example, as anti-viral, anti-cancer or anti-parasitic drugs.

As used herein, the terms “lipophilic” or “long-chain” refer to the cyclic, branched or straight chain chemical groups that when covalently linked to a phosphonic acid to form a phosphonate monoester increase oral bioavailability and enhance activity of the nucleoside phosphonates as compared with the parent nucleoside phosphonates. These lipophilic groups include, but are not limited to alkyl, alkoxyalkyl, and alkylglyceryl.

As used herein, the term “lipophilic monoesters of nucleoside phosphonates” refers to compound where a lipophilic group is covalently attached to a nucleoside phosphonate via an ester linkage.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, and cycloalkyl. Pharmaceutically acceptable enol esters include, but are not limited to, derivatives of formula C═C(OC(O)R) where R is hydrogen, alkyl, alkenyl, alkynyl, or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392). Other prodrugs for use herein are described elsewhere herein.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the compounds provided herein encompass any racemic, optically active, polymorphic, or steroisomeric form, or mixtures thereof, of a compound provided herein, which possesses the useful properties described herein, it being well known in the art how to prepare optically active forms and how to determine antiproliferative activity using the standard tests described herein, or using other similar tests which are well known in the art. Examples of methods that can be used to obtain optical isomers of the compounds provided herein include the following:

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or

complete separation of a racemate by virtue of differing rates of reaction for the enantiomers with an enzyme

iv) enzymatic asymmetric synthesis—a synthetic technique

whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic

compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a

technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer,

viii) kinetic resolutions—this technique refers to the

achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, the term “alkyl” refers to a monovalent straight or branched chain or cyclic radical. In certain embodiments, the alkyl group contains from one to twenty-four carbon atoms, including methyl, ethyl, n-propyl, isopropyl, n-butyl, isobutyl, tert-butyl, n-hexyl, octadecyl, nonadecyl, eicosyl, 18-methyl-nonadecyl, 19-methyl-eicosyl, and the like. As used herein lower alkyl refers to alkyl groups of 1 to 6 carbon atoms.

As used herein, “substituted alkyl” refers to alkyl groups further bearing one or more substituents, including, but not limited to substituents selected from lower alkyl, hydroxy, alkoxy (of a lower alkyl group), mercapto (of a lower alkyl group), cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, azido, nitro, nitrone, amino, amido, —C(O)H, acyl, oxyacyl, carboxyl, carbamate, sulfonyl, sulfonamide, and sulfuryl, which may be protected or unprotected as necessary, as taught in Greene, et al., Protective Groups in Organic Synthesis, John Wiley and Sons, Second Ed. 1991, hereby incorporated by reference.

As used herein, “alkenyl” refers to straight or branched chain hydrocarbon group having one or more carbon-carbon double bonds. In certain embodiments, the alkenyl group contains from 2 up to 24 carbon atoms, and “substituted alkenyl” refers to alkenyl groups further bearing one or more substituents as set forth above.

As used herein, “alkynyl” refers to straight or branched chain hydrocarbon group having one or more carbon-carbon triple bonds. In certain embodiments, the alkynyl group contains from 2 up to 24 carbon atoms, and “substituted alkynyl” refers to alkynyl groups further bearing one or more substituents as set forth above.

As used herein, “aryl” refers to aromatic groups having in the range of 6 up to 14 carbon atoms and “substituted aryl” refers to aryl groups further bearing one or more substituents as set forth above.

As used herein, “heteroaryl” refers to aromatic groups containing one or more heteroatoms (e.g., N, O, S, or the like) as part of the ring structure, and having in the range of 3 up to 14 carbon atoms and “substituted heteroaryl” refers to heteroaryl groups further bearing one or more substituents as set forth above.

As used herein “subject” is an animal, such as a mammal, including human, such as a patient.

The phrase “effective amount” as used herein means an amount required for prevention, treatment, or amelioration of one or more of the symptoms of diseases or disorders associated including those associated with viral infection, cell proliferation and/or bone metabolism.

Where the number of any given substituent is not specified (e.g., haloalkyl), there may be one or more substituents present. For example, “haloalkyl” may include one or more of the same or different halogens.

As used herein, the term “parenteral” includes subcutaneous, intravenous, intra-arterial, intramuscular or intravitreal injection, or infusion techniques.

The term “topically” encompasses administration rectally and by inhalation spray, as well as the more common routes of the skin and mucous membranes of the mouth and nose and in toothpaste.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

Some abbreviations used herein are as follows:
Hexadecyloxypropyl=HDP
Octadecyloxyethyl=ODE
Oleyloxyethyl=OLE,
Oleyloxypropyl=OLP,
(S)-9-[3-hydroxy-2-(phosphonomethoxy)-propyl]cytosine=HPMPC (cidofovir),
(S)-9-[3-hydroxy-2-(phosphonomethoxy)-propyl]adenine=(S)-HPMPA,
Phosphonomethoxyethyl-guanine=PMEG,
Phosphonomethoxyethyl-adenine=PMEA,
Phosphonomethoxy-propyladenine=PMPA (tenofovir),
Hexadecyloxypropyl-phospho-(S)-9-[3-hydroxy-2-(phosphonomethoxy)-propyl]adenine=HDP-phospho-(S)-HPMPA,
Octadecyloxyethyl-phospho-(S)-9-[3-hydroxy-2-phosphonomethoxy)-propyl]adenine=ODE-phospho-(S)-HPMPA,
Oleyloxyethyl-phospho-(S)-9-[3-hydroxy-2-(phosphonomethoxy)-propyl]adenine=OLE-phospho-(S)-HPMPA,
Oleyloxypropyl-phospho-(S)-9-[3-hydroxy-2-(phosphonomethoxy)-propyl]adenine=OLP-phospho-(S)-HPMPA,
5-Phosphono-pent-2-en-1-yl adenine=PPen-A,
5-Phosphono-pent-2-en-1-yl cytosine=PPen-C,
5-Phosphono-pent-2-en-1-yl guanine=PPen-G,
5-Phosphono-pent-2-en-1-yl thymine=PPen-T and
5-Phosphono-pent-2-en-1-yl uracil=PPen-U.

### B. Compounds

In certain embodiments, the compound for use in the compositions and methods provided herein has formula II:

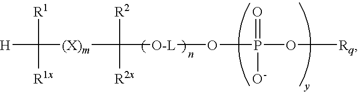

or a pharmaceutically active derivatives thereof,
wherein;
R¹ and R^{1x} are each independently —H, optionally substituted —O(C₁-C₂₄)alkyl, —O(C₁-C₂₄)alkenyl, —O(C₁-C₂₄)acyl, —S(C₁-C₂₄)alkyl, —S(C₁-C₂₄)alkenyl, or —S(C₁-C₂₄)acyl, wherein at least one of R¹ and R^{1x} is not —H, and wherein the alkenyl or acyl optionally have 1 to about 6 double bonds;
R² and R^{2x} are each independently —H, optionally substituted —O(C₁-C₇)alkyl, —O(C₁-C₇)alkenyl, S(C₁-C₇)alkyl, —S(C₁-C₇)alkenyl, —O(C₁-C₇)acyl, —S(C₁-C₇)acyl, —N(C₁-C₇)acyl, NH(C₁-C₇)alkyl, —N((C₁-C₇)alkyl)₂, halogen, —NH₂, —OH, or —SH;
R_{q} is a pharmacologically active phosphonate or a phosphonate derivative of a pharmacologically active compound, coupled to the phosphate group by an anhydride linkage;
X, when present, is:

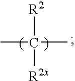

L is a valence bond or a bifunctional linking molecule of the formula -J-(CR^{x}R^{y}N-G-, wherein t is an integer from 1 to 24; J and G are independently —, —S—, —C(O)O— or —NH—; R^{x} and R^{y} are each independently —H, substituted or unsubstituted alkyl, or alkenyl;

m is an integer from 0 to 6; y is 1 or 2;

and n is 0 or 1.

In certain embodiments, the compounds for use in the compositions and methods provided herein have formula III:

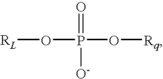

or pharmaceutically active derivatives thereof,

wherein R_{q} is a pharmacologically active phosphonate or a phosphonate derivative of a pharmacologically active compound and R_{q} has formula:

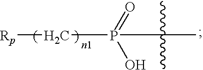

wherein Rₚ is a pharmacologically active nucleoside or an analog thereof.
In certain embodiments, in the compounds of formula II,
R¹ and R^{1x} are each independently —H, or optionally substituted —O(C₁-C₂₄)alkyl; wherein at least one of R¹ and R^{1x} is not —H;
R² and R^{2x} are each independently —H, or optionally substituted —O(C₁-C₇)alkyl;
R_{q} is a pharmacologically active phosphonate or a phosphonate derivative of a pharmacologically active compound of formula:

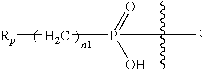

Rₚ is a pharmacologically active nucleoside or analog thereof;

L is a valence bond or a bifunctional linking molecule of the formula -J-(CR^{x}R^{y})ₜ-G-, wherein t is an integer from 1 to 24, J and G are each independently —, and R^{x} and R^{y} are each independently —H, substituted or unsubstituted alkyl, or alkenyl;

n is 0 or 1; and

n′ is 0 to 3.

In certain embodiments, Rₚ is

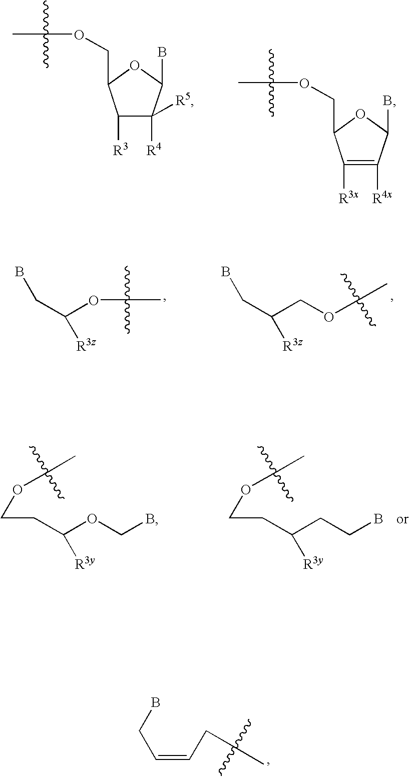

wherein
R³, R⁴ and R⁵ are each independently H, hydroxy, halo, azido, C₁₋₆ alkyl, C₂₋₆ alkenyl or C₂₋₆ alkynyl;
B is a purine or pyrimidine base or an analog thereof;
R^{3x} is H, azido, substituted or unsubstituted C₁₋₆ alkyl, substituted or unsubstituted C₂₋₆ alkenyl or substituted or unsubstituted C₂₋₆ alkynyl;
R^{4x} is H, C₁₋₆ substituted or unsubstituted alkyl, C₂₋₆ substituted or unsubstituted alkenyl or C₂₋₆ substituted or unsubstituted alkynyl; and
R^{3z} is H, C₁₋₆ alkyl, hydroxylCl₄₋₆ alkyl, haloC₁₋₆ alkyl, azidoC₁₋₆ alkyl or OH.
In certain embodiments, Rₚ has formula:

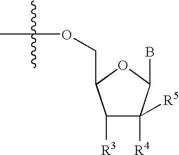

wherein the variables are as described elsewhere herein.

In certain embodiments, R³ is H, azido, substituted or unsubstituted C₁₋₆ alkyl, substituted or unsubstituted C₂₋₆ alkenyl or substituted or unsubstituted C₂₋₆ alkynyl. In certain embodiments, R³ is H, azido, substituted or unsubstituted C₁₋₆ alkyl. In certain embodiments, R³ is H or azido. In certain embodiments, R³ is azido. In certain embodiments, R³ is H. In certain embodiments, R⁴ and R⁵ are each independently selected from hydrogen, halo and hydroxyalkyl. In certain embodiments, R⁴ and R⁵ are each independently selected from halo and hydroxyalkyl. In certain embodiments, R⁴ and R⁵ are each independently selected from fluoro and hydroxymethyl. In certain embodiments, R⁴ is selected from fluoro and hydroxymethyl. In certain embodiments, R⁵ is selected from fluoro and hydroxymethyl.
In certain embodiments, Rₚ has formula:

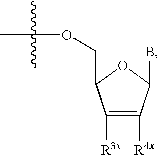

wherein the variables are as described elsewhere herein.

In certain embodiments, R^{3x} is H, azido, substituted or unsubstituted C₁₋₆ alkyl, substituted or unsubstituted C₂₋₆ alkenyl or substituted or unsubstituted C₂₋₆ alkynyl; R^{4x} is H, C₁₋₆ substituted or unsubstituted alkyl, C₂₋₆ substituted or unsubstituted alkenyl or C₂₋₆ substituted or unsubstituted alkynyl and other variables are as defined elsewhere herein. In certain embodiments, R^{3x} is H, azido or substituted or unsubstituted C₁₋₆ alkyl. In certain embodiments, R^{4x} is H, C₁₋₆ substituted or unsubstituted alkyl, C₂₋₆ substituted or unsubstituted alkenyl or C₂₋₆ substituted or unsubstituted alkynyl. In certain embodiments, R^{4x} is H, or C₁₋₆ alkyl. In certain embodiments, R^{4x} is H, or methyl.

In certain embodiments, Rₚ has formula:

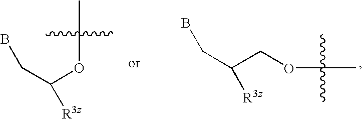

wherein the variables are as described elsewhere herein.

In certain embodiments, R^{3e} is H, C₁₋₆ alkyl, hydroxyl C₁₋₆ alkyl, halo C₁₋₆ alkyl, azido C₁₋₆ alkyl or OH and the other variables are as defined elsewhere herein. In certain embodiments, R^{3z} is hydrogen, C₁₋₆ alkyl or hydroxyC₁₋₆ alkyl. In certain embodiments, R^{3z} is hydrogen or hydroxymethyl. In certain embodiments, R^{3z} is hydrogen. In certain embodiments, R^{3z} is hydroxymethyl. In certain embodiments, the OH group is protected, for example as an ester or an ether. In certain embodiments, R^{3z} may be in S or R configuration.

In certain embodiments, Rₚ has formula:

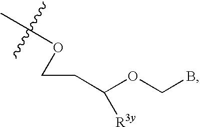

wherein the variables are as described elsewhere herein.

In certain embodiments, R^{3y} is H, substituted or unsubstituted C₁₋₆ alkyl, substituted or unsubstituted C₂₋₆ alkenyl or substituted or unsubstituted C₂₋₆ alkynyl, or OH and the other variables are as defined elsewhere herein. In certain embodiments, R^{3y} is hydrogen, C₁₋₆ alkyl or hydroxyl C₁₋₆ alkyl. In certain embodiments, R^{3y} is hydrogen or hydroxymethyl. In certain embodiments, R^{3y} may be in S or R configuration.

In certain embodiments, Rₚ has formula:

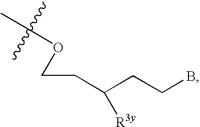

wherein the variables are as described elsewhere herein.

In certain embodiments, Rₚ has formula:

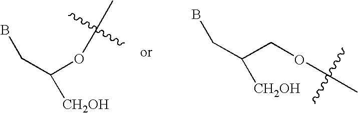

In certain embodiments, m=0, 1 or 2. In certain embodiments, m=0 or 1. In certain embodiments, m=0. In certain embodiments, m=1. In certain embodiments, R² and R^{2x} are H.

In certain embodiments, R_{L} has formula:

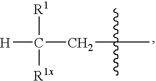

wherein R¹ and R^{1x} are as defined elsewhere herein.

In certain embodiments, R_{L} has formula:

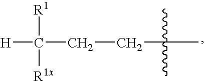

wherein R¹ and R^{1x} are as defined elsewhere herein.

In certain embodiments, R_{L} has formula:

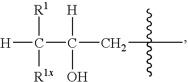

wherein R¹ and R^{1x} are as defined elsewhere herein.

In certain embodiments, R_{L} is hexadecyloxypropyl, octadecyloxypropyl, oleyloxyethyl, oleyloxypropyl, octadecyloxyethyl, 15-methylhexadecyloxypropyl or 17-methyloctadecyloxyethyl.

In certain embodiments, R¹ is an alkoxy group having the formula —O—(CH₂)ₜ—CH₃ wherein t is 0-24. In other embodiments, t is 8, 10, 12, 13, 14, 15, 16, 17, 18, 19 or 20. In other embodiments, t is 13, 14, 15, 16, 17, 18, 19 or 20. In other embodiments, t is 15, 16, 17, 18, 19 or 20. In other embodiments, t is 17, 18, 19 or 20. In other embodiments, t is 15 or 17.

In certain embodiments, R_{L} is a substituted or unsubstituted C₈-C₂₄ alkyl, substituted or unsubstituted C₈-C₂₄ alkenyl having from 1 to 6 double bonds or substituted or unsubstituted C₈-C₂₄ alkynyl having from 1 to 6 triple bonds, wherein substituents when present are selected from one or more, in some embodiments, 1 to 4 or 1 or 2 substituents selected from halogen, alkyl, —OR^{w}, —SR^{w}, cycloalkyl or epoxide, where R^{w} is hydrogen or alkyl and where the alkyl, alkenyl, alkynyl groups may be further substituted or unsubstituted.

In certain embodiments, R_{L} is an alkyl, alkenyl or alkynyl group and contains 8, 10, 12, 13, 14, 15, 16, 17, 18, 19, 20, 21, 22, 23 or 24 carbon atoms and can be a straight or branched chain moiety. In certain embodiments, R_{L} is a C₁₆-C₂₃ straight or branched chain alkyl or C₁₆-C₂₃ straight or branched chain alkenyl. In other embodiments, R_{L} is a C₁₇-C₁₉ straight or branched chain alkyl or C₁₇-C₁₉ straight or branched chain alkenyl. In other embodiments, R_{L} is C₁₇-alkyl, C₁₈-alkyl or C₁₉ alkyl. In other embodiments, R_{L} is C₁₇-alkenyl, C₁₈-alkenyl or C¹⁹ alkenyl. In other embodiments, R_{L} is C₁₇-C₂₂ alkyl. In other embodiments, R_{L} is C₁₇ alkyl, C₁₈ alkyl, C₁₉ alkyl, C₂₀ alkyl, C₂₁ alkyl, or C₂₂ alkyl.

In certain embodiments, R_{L} is substituted with one or more groups selected from lower alkyl and halo. In certain embodiments, R_{L} is substituted with one or more methyl groups. In certain embodiments, R_{L} is substituted with one or more fluoro groups. In certain embodiments, R_{L} is C₁₆-C₂₃ alkyl and is substituted with one or more methyl or fluoro groups. In certain embodiments, the methyl group or the fluoro group substituent is present on the penultimate carbon of the alkyl, alkenyl, or alkynyl chain. In certain embodiments, R_{L} is 7-methyl-octyl, 8-methyl-nonyl, 9-methyl-decyl, 10-methyl-undecyl, 11-methyl-dodecyl, 12-methyl-tridecyl, 13-methyl-tetradecyl, 14-methyl-pentadecyl, 15-methyl-hexadecyl, 16-methyl-heptadecyl, 17-methyl-octadecyl, 18-methyl-nonadecyl, 19-methyl-eicosyl, 20-methyl-heneicosyl, 21-methyl-docosyl, 122-methyl-tricosyl, 7-fluoro-octyl, 8-fluoro-nonyl, 9-fluoro-decyl, 10-fluoro-undecyl, 11-fluoro-dodecyl, 12-fluoro-tridecyl, 13-fluoro-tetradecyl, 14-fluoro-pentadecyl, 15-fluoro-hexadecyl, 16-fluoro-heptadecyl, 17-fluoro-octadecyl, 18-fluoro-nonadecyl, 19-fluoro-eicosyl, 20-fluoro-heneicosyl, 21-fluoro-docosyl or 22-fluoro-tricosyl.

In certain embodiments, B is selected from a natural or non natural purine or pyrimidine base. In certain embodiments, B is

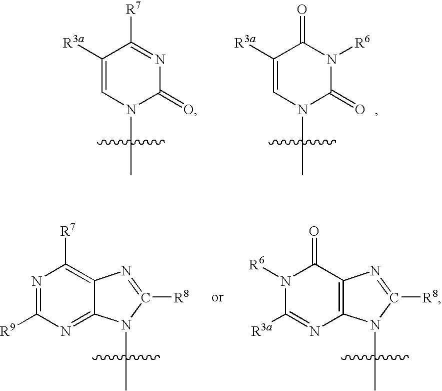

wherein R^{3a} is H, C₁₋₆ alkyl, C₂₋₆ alkenyl, C₂₋₆ alkynyl, C₃₋₆ cycloalkyl, hydroxy, halo, aryl or heteroaryl;

R⁶ is H or C₁₋₆ alkyl, C₂₋₆ alkenyl, C₂₋₆ alkynyl or cycloalkyl;

R⁷ is H, hydroxy, halo, C₁₋₆ alkyl, C₂₋₆ alkenyl, C₂₋₆ alkynyl, cycloalkyl or NR⁴R⁵;

R⁸ is H, C₁₋₆ alkyl, C₂₋₆ alkenyl, C₂₋₆ alkynyl or cycloalkyl; and

R⁹ is H, C₁₋₆ alkyl, C₂₋₆ alkenyl, C₂₋₆ alkynyl, cycloalkyl, halo or NR⁴R⁵, where R⁴ and R⁵ are each independently H, C₁₋₆ alkyl, C₂₋₆ alkenyl, C₂₋₆ alkynyl, or C₃₋₆ cycloalkyl.

In other embodiments, R^{3a} is H, C₁₋₆ alkyl, C₂₋₆ alkenyl, C₂₋₆ alkynyl, C₃₋₆ cycloalkyl, hydroxy, halo, aryl or heteroaryl. In other embodiments, R^{3a} is H, halo or C₁₋₆ alkyl. In other embodiments, R^{3a} is H. In other embodiments, R^{3a} is methyl. In other embodiments, R^{3a} is fluoro.

In other embodiments, R⁴ and R⁵ are each independently H, C₁₋₆ alkyl, C₂₋₆ alkenyl, C₂₋₆ alkynyl, or C₃₋₆ cycloalkyl. In other embodiments, R⁴ is H, C₁₋₆ alkyl or C₃₋₆ cycloalkyl. In other embodiments, R⁴ is H, methyl or cyclopropyl. In other embodiments, R⁵ is H, C₁₋₆ alkyl or C₃₋₆ cycloalkyl. In other embodiments, R⁵ is H, methyl or cyclopropyl.

In other embodiments, R⁶ is H or C₁₋₆ alkyl, C₂₋₆ alkenyl, C₂₋₆ alkynyl or cycloalkyl. In other embodiments, R⁶ is H or C₁₋₆ alkyl. In other embodiments, R⁶ is H or methyl. In other embodiments, R⁶ is H. In other embodiments, R⁶ is methyl.

In other embodiments, R⁷ is H, hydroxy, halo, C₁₋₆ alkyl, C₂₋₆ alkenyl, C₂₋₆ alkynyl, cycloalkyl or NR⁴R⁵. In other embodiments, R⁷ is H, C₁₋₆ alkyl, or NR⁴R⁵. In other embodiments, R⁷ is methyl. In other embodiments, R⁷ is NR⁴R⁵. In other embodiments, R⁷ is NH₂.

In other embodiments, R^{a} is H, C₁₋₆ alkyl, C₂₋₆ alkenyl, C₂₋₆ alkynyl or cycloalkyl. In other embodiments, R⁸ is H.

In other embodiments, R⁹ is H, C₁₋₆ alkyl, C₂₋₆ alkenyl, C₂₋₆ alkynyl, cycloalkyl, halo or NR⁴R⁵. In other embodiments, R⁹ is H.

In other embodiments, B is selected from pyrimidin-1-yl, pyrimidin-3-yl, purin-3-yl, purin-7-yl and purin-9-yl residue. In certain embodiments, B is thymin-1-yl, cytosine-1-yl, adenine-9-yl or guanine-9-yl.

In other embodiments, B is selected from:

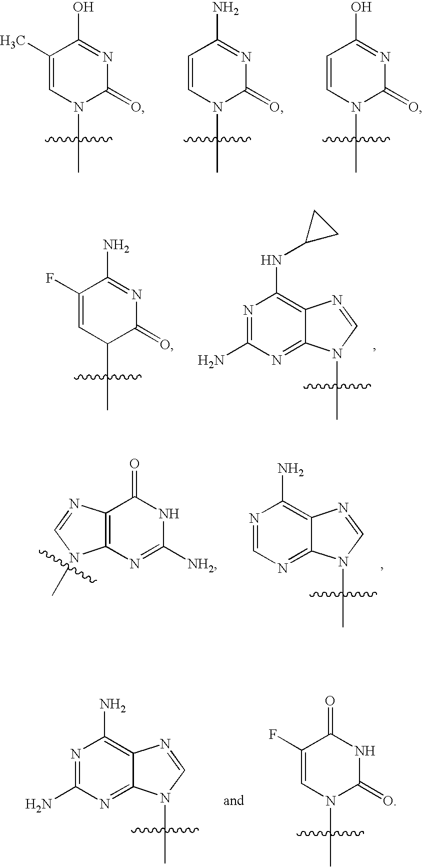

In certain embodiments, the alkyl, alkenyl and alkynyl groups in the compounds provided herein are substituted with one or more, in one embodiment, one, two, three or four substituents selected from alkyl, alkenyl, alkynyl, halo, hydroxyl, pseudohalo, amino, nitro, cycloalkyl, heterocyclyl, aryl and heteroaryl.

Exemplary Compounds

In certain embodiments, the compounds herein are phosphate esters of antiviral and anticancer nucleoside phosphonates. In certain embodiments, the compounds provided herein are analogs of (S)-9-[3-hydroxy-2-(phosphonomethoxy)-propyl]cytosine (HPMPC, cidofovir), (S)-9-[3-hydroxy-2-(phosphonomethoxy)-propyl]adenine ((S)-HPMPA), phosphonomethoxyethyl-guanine (PMEG), phosphonomethoxyethyl-adenine (PMEA) and phosphonomethoxy-propyladenine (PMPA, tenofovir). Many other acyclic nucleoside phosphonates can be modified by conjugation to alkoxyalkyl-phosphate and alkyl-phosphates as described herein. Certain exemplary compounds that can be modified as provided herein are described in the documents listed in Table 1. All the documents listed herein are hereby incorporated by reference in their entirety.

TABLE 1

| Document | | Author |
|---|---|---|
| ID | Title | (first) |
| CZ 292199 | O-Phosphonomethyl Choline and Alkyl | Holy, A. |
| | Esters thereof, process of their | (anti-cancer agents) |
| | preparation and use | |
| U.S. Pat. No. 6,653,296 | Preparation of Anti-retroviral | Holy, A. |
| U.S. Pat. No. 6,057,305 | Enantiomeric nucleotide Analogs | (tenofovir, PMPA) |
| U.S. Pat. No. 5,977,061 | Acyclic Nucleosides as Virucides and | Holy, A. |
| | Immunostimulation Suppressants | (HPMPDAP) |
| U.S. Pat. No. 5,733,896 | Preparation of N-(3-fluoro-2- | Holy, A. |
| | phosphonylmethoxypropyl)purines and - | |
| | pyrimidines as antiviral agents | |
| CZ 263953 | Method for the Preparation of 9-(S)- and | Holy, A. |
| | 9-(R,S)-(3-hydroxy-2- | |
| | phosphonylmethoxy propyl)adenine as | |
| | virucides | |
| CZ 263955 | Method for the preparation of N-[3- | Holy, A. |
| | hydroxy-2- | |
| | (phosphonylmethoxy)propyl]purines | |
| | and -pyrimidines | |
| CZ 263956 | Method for the preparation of virucidal | Holy, A. |
| | 9-(S)-(3-hydroxy-2- | |
| | phosphonylmethoxypropyl)adenine | |
| U.S. Pat. No. 5,641,763 | Preparation and testing of N- | Holy, A. |
| U.S. Pat. No. 5,869,467 | phosphonylmethoxyalkyl derivatives of | (excludes HPMPA, includes |
| | pyrimidine and purine bases with | PMEG) |
| | antiviral activity | |
| U.S. Pat. No. 4,808,716 | Preparation of 9- | Holy, A. |
| | [phosphonomethoxy)alkyl]adenines and | (HPMPA) |
| | their use as virucides | |
| U.S. Pat. No. 4,724,233 | Use of | De Clerq, E. |
| | phosphonylmethoxyalkyladenines in the | |
| | treatment of virus diseases | |
| FR 2539132 | Isomeric O-phosphonylmethyl | Holy, A. |
| | derivatives of enantiomeric and racemic | |
| | vicinal diols | |
| WO 2004096286 | Preparation of phosphonate prodrugs of | Boojamara, C. G. |
| | antiviral compounds | |
| US 2004023928 | Phosphonate nucleotide and thiadiazole | Colacino, J. M. |
| | compounds for the treatment of smallpox | |
| US 2004023921 | Antiviral Phosphonate Nucleotide | Hong, Z. |
| | Analogs | |
| WO 2003099294 | Improvement in drug selectivity of | Ubasawa, K. |
| | targeting tissues for therapeutic | |
| | efficiency | |
| WO 2003090691 | Preparation of phosphonate analogs of | Birkus, G. |
| | HIV protease inhibitors and methods for | |
| | identifying anti-HIV therapeutic | |
| | compounds | |
| WO 2003090690 | Preparation of phosphonate analogs of | Arimilli, M. N. |
| | HIV protease inhibitors with improved | |
| | cellular accumulation properties | |
| WO 2003050129 | Use of phosphonate nucleotide analog | Wise, S. D. |
| | LY582563 for treating hepatitis B virus | |
| | infections | |
| US 2003109498 | 2-Amino-6-arylthiopurine phosphonate | Yuasa, S. |
| | antiviral agents for treatment of drug- | |
| | resistant virus infections | |
| RU 2187509 | Preparation of derivatives of 3′-azido-3′- | Shirokova, E. A. |
| | deoxythymidine 5′-H-phosphonates as | |
| | antiviral agents | |
| WO 2003002580 | Preparation of phosphonate-substituted | Balzarini, J. M. |
| | pyrimidine analogs as antiviral agents | (DAPy) |
| US 644656 | Preparation of antiviral phosphonate | Nguyen-Ba, Nghe |
| U.S. Pat. No. 5,955,610 | nucleotides | |
| U.S. Pat. No. 6,005,107 | | |
| U.S. Pat. No. 6,127,540 | | |
| WO 2002057288 | Preparation of acyclic nucleoside | Choi, J-R. |
| | phosphonates as antiviral agents against | |
| | hepatitis B virius | |
| WO 2001064693 | Preparation of phosphonate nucleotide | Ubasawa, M. |
| | compounds as antiviral agents | |
| WO 2000029414 | Preparation of dialkyl 1-aryl-1-[(2,4- | Tets, V. |
| | dioxo-1,3-pyrimidin-5- | |
| | yl)amino]methylphosphonates as | |
| | antiviral and antimicrobial agents | |
| WO 9962921 | Preparation of antiviral phosphorous | Alexandrovna, A. |
| | derivatives of 4-thio-5-ethyl-2′- | |
| | deoxyuridine | |
| U.S. Pat. No. 5,886,179 | Preparation of nucleotide phosphonate | Arimilli, M. N. |
| U.S. Pat. No. 5,656,745 | ester analogs as antiviral agents | |
| WO 9319075 | Preparation of purine-substituted | Harnden, M. R. |
| | phosphonates as antiviral agents | |
| U.S. Pat. No. 5,817,647 | Unsaturated phosphonate derivatives of | Casara, P. |
| U.S. Pat. No. 5,922,696 | purines and pyrimidines | |
| U.S. Pat. No. 5,532,225 | Preparation of acyclic purine | Reist, E. J. |
| | phosphonate nucleotide analogs as | |
| | antiviral agents | |
| U.S. Pat. No. 5,877,166 | Preparation of enantiomerically pure 2- | Reist, E. J. |
| | aminopurine phosphonate nucleotide | |
| | analogs as antiviral agents | |
| WO 9842351 | Preparation of difluoronucleoside | Hertel, L. W. |
| | phosphonic acids as antiviral and | |
| | antineoplastic agents | |
| WO 9838202 | Preparation of nucleoside phosphonates | Rosowsky, A. |
| | as antivirals | |
| U.S. Pat. No. 5,717,095 | Preparation of cyclic nucleotide | Arimilli, M. N. |
| | phosphonate esters as virucides | |
| U.S. Pat. No. 5,650,510 | Antiviral | Webb, R. R. |
| U.S. Pat. No. 5,854,228 | phosphonomethoxyalkylpurines and - | (PMEG) |
| | pyrimidines and their preparation | |
| U.S. Pat. No. 5,840,716 | Preparation of acyclic nucleotide | Ubasawa, M. |
| | phosphonates as vinicides | |
| U.S. Pat. No. 5,798,340 | Preparation of virucidal nucleotide | Bischofberger, N. W. |
| U.S. Pat. No. 6,225,460 | analogs | |
| U.S. Pat. No. 6,197,775 | Prepararation of phosphorate acyclic | Ubasawa, M. |
| | nucleotide derivatives as antiviral agents | |

Nucleoside analogs with antiviral activity against hepatitis C may be converted to their 5′-phosphonates or 5′-methylene phosphonates for use in the compounds provided herein. Some exemplary nucleosides include: 2′-C-methyl adenosine; 2′-C-methyl guanosine; 7-deaza-2′-C-methyl adenosine, 2′-C-methyl cytosine. Other nucleosides which can be used in the compounds provided herein, after conversion of their 5′-phosphonates or 5′-methylene-phosphonates are described in the following patents, which are hereby incorporated by reference in their entirety.

TABLE 1a

| Document | | Author |
|---|---|---|
| ID | Title | (first) |
| US 2003/0050229 A1 | Methods and compositions for | Sommadossi, |
| | treating hepatitis C virus | J-P |
| US 2003/0060400 A1 | Methods and compositions for | LaColla, P. |
| | treating flaviviruses and | |
| | pestiviruses | |
| US 2003/0087873 A1 | Modified nucleosides for | Stuyver, L. |
| | treatment of viral infections | |
| | and abnormal cell proliferation | |
| US 2004/0063622 A1 | Methods and compositions for | Sommadossi, |
| | treating flaviviruses and | J-P |
| | pestiviruses | |
| US 2004/0067877 A1 | 2′,3-dideoxynucleosides for | Schinazi, R. F. |
| | prevention or treatment of | |
| | flaviviridae infections | |
| US 2004/0097461 A1 | Methods and compositions for | Sommadossi, |
| | treating hepatitis C virus | J-P. |
| US 2004/0097462 A1 | Methods and compositions for | Sommadossi, |
| | treating flaviviruses and | J-P. |
| | pestiviruses | |
| US 2004/0101535 A1 | Methods and compositions for | Sommadossi, |
| | treating hepatitis C virus | J-P. |
| US 2004/0254141 A1 | 2′-fluoronucleosides | Schinazi, R. F. |
| US 2003/0008841 A1 | Anti-HCV Nucleoside | Devos, R. |
| | Derivatives | |
| US 2002/0055483 A1 | 3′- or 2-hydroxymethyl | Watanabe, |
| | substituted nucleoside | K. A. |
| | derivatives for treatment of | |
| | hepatitis virus infections | |
| US 2002/0147160 A1 | Nucleoside derivatives as | Bhat, B. |
| | inhibitors of RNA-dependent | |
| | RNA viral polymerase | |
| US 2005/0009775 A1 | Nucleoside compounds in hcv | Howes, P. D. |
| US 2005/0009737 A1 | Modified fluorinated nucleosides | Clark, J. |
| US 20040266722 A1 | 4′-substituted nucleosides as | Devos, R. |
| | inhibitors of HCV RNA | |
| | replication | |

Nucleoside analogs with antiviral activity against hepatitis B may be converted to their 5′-phosphonates or 5′-methylene phosphonates for use herein. Exemplary nucleosides include 3TC, FTC, DAPD, L-FMAU, entecavir, telbivudine and various β-L-2′-deoxycytidine, β-L-2′-deoxyadenine and β-L-2′-deoxythymidine analogs described by Bryant et al., Anti-viral L-nucleosides specific for Hepatitis B infection, Antimicrob. Agents Chemother., 45:229-235, 2001. In certain embodiments, the nucleosides for use herein include, but are not limited to tenofovir, adefovir, and the 5-phosphono-pent-2-en-1-yl nucleosides, such as 5-phosphono-pent-2-en-1-yl adenine (PPen-A), 5-phosphono-pent-2-en-1-yl cytosine (PPen-C), 5-phosphono-pent-2-en-1-yl guanine (PPen-G), 5-phosphono-pent-2-en-1-yl thymine (PPen-1) and 5-phosphono-pent-2-en-1-yl uracil (PPen-U) and others disclosed in U.S. Application Ser. No. 60/667,740, which incorporated by reference in its entirety. In certain embodiments, the compounds have anti-hepatitis B activity. Certain other Nucleoside 5′-monophosphates for use herein are described by Prakash et al. in J. Med. Chem. 2005, 48, 1199-1210.

In certain embodiments, the compounds provided herein are selected from hexadecyloxypropyl-phospho-(S)-HPMPA (HDP-phospho-(S)-HPMPA), octadecyloxyethyl-phospho-(S)-HPMPA (ODE-phospho-(S)-HPMPA), oleyloxyethyl-phospho-(S)-HPMPA (OLE-phospho-(S)-HPMPA), oleyloxypropyl-phospho-(S)-HPMPA (OLP-phospho-(S)-HPMPA), 15-methyl-hexadecyloxy-propyl-phospho-(S)-HPMPA, and 17-methyl-octadecyloxy-ethyl-phospho-(S)-HPMPA.

Additional description of the compounds provided herein, including general structural formulas are given Table 2.

TABLE 2

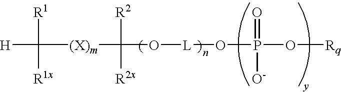

| Compound | y | R¹ | R^{1x} | X | m | R² | R^{2x} | L | n | Rq |
|---|---|---|---|---|---|---|---|---|---|---|
| HDP-phospho- (S)-HPMPA | 1 | CH₃(CH₂)₁₅O | H | CH₂ | 1 | H | H | absent | 0 | 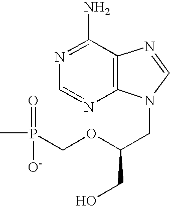 |
| ODE-phospho- (S)-HPMPA | 1 | CH₃(CH₂)₁₇O | H | absent | 0 | H | H | absent | 0 | 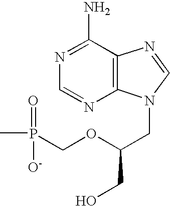 |
| OLE-phospho- (S)-HPMPA | 1 | CH₃(CH₂)₇CH═CH—(CH₂)₈O | H | absent | 0 | H | H | absent | 0 | 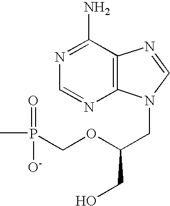 |
| OLP-phospho- (S)-HPMPA | 1 | CH₃(CH₂)₇CH═CH—(CH₂)₈O | H | CH₂ | 1 | H | H | absent | 0 | 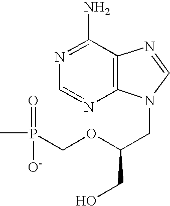 |
| 15-methyl- hexadecyloxy- propyl- phospho-(S)- HPMPA | 1 | CH₃CH(CH₃)(CH₂)₁₄O | H | CH₂ | 1 | H | H | absent | 0 | 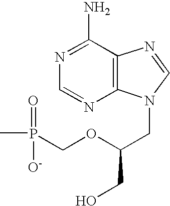 |
| 17-methyl- octadecyloxy- ethyl-phospho- (S)-HPMPA | 1 | CH₃CH(CH₃)(CH₂)₁₆O | H | absent | 0 | H | H | absent | 0 | 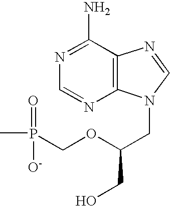 |
| 16-fluoro- hexadecyloxy- propyl- phospho-(S)- HPMPA | 1 | CH₂(F)(CH₂)₁₅O | H | CH₂ | 1 | H | H | absent | 0 | 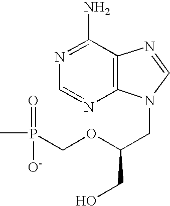 |
| 18-fluoro- octadecyloxy-ethyl- phospho-(S)-HPMPA | 1 | CH₂(F)(CH₂)₁₇O | H | absent | 0 | H | H | absent | 0 | 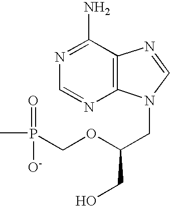 |
| 15-methyl- hexadecyloxy- ethyl-phospho- (S)-HPMPA | 1 | CH₃CH(CH₃)(CH₂)₁₄O | H | absent | 0 | H | H | absent | 0 | 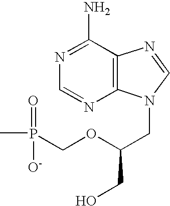 |
| HDP-phospho- cidofovir | 1 | CH₃(CH₂)₁₅O | H | CH₂ | 1 | H | H | absent | 0 | 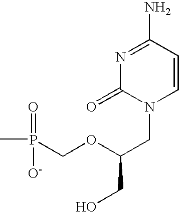 |
| ODE-phospho- cidofovir | 1 | CH₃(CH₂)₁₇O | H | absent | 0 | H | H | absent | 0 | 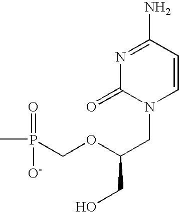 |
| OLE-phospho- cidofovir | 1 | CH₃(CH₂)₇CH═CH—(CH₂)₈O | H | absent | 0 | H | H | absent | 0 | 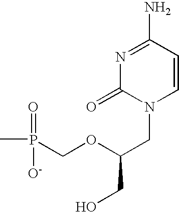 |
| OLP-phospho- cidofovir | 1 | CH₃(CH₂)₇CH═CH—(CH₂)₈O | H | CH₂ | 1 | H | H | absent | 0 | 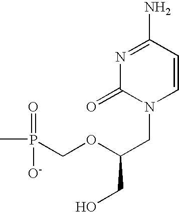 |
| HDP-phospho- PMEG | 1 | CH₃(CH₂)₁₅O | H | CH₂ | 1 | H | H | absent | 0 | 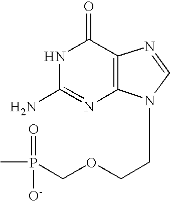 |
| ODE-phospho- PMEG | 1 | CH₃(CH₂)₁₇O | H | absent | 0 | H | H | absent | 0 | 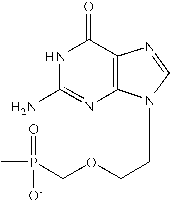 |
| HDP-phospho- PME-DAP | 1 | CH₃(CH₂)₁₅O | H | CH₂ | 1 | H | H | absent | 0 | 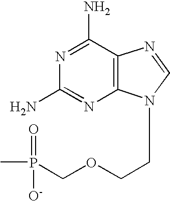 |
| HDP-phospho- PME-N⁶cPr- DAP | 1 | CH₃(CH₂)₁₅O | H | CH₂ | 1 | H | H | absent | 0 | 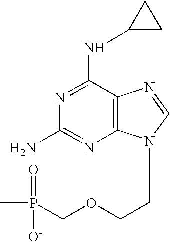 |
| OLE-phospho- PME-N⁶cPr- DAP | 1 | CH₃(CH₂)₇CH═CH—(CH₂)₈O | H | absent | 0 | H | H | absent | 0 | 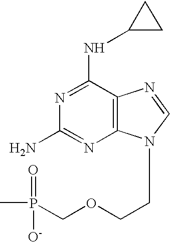 |
| HDP-phospho- PPMG | 1 | CH₃(CH₂)₁₅O | H | CH₂ | 1 | H | H | absent | 0 | 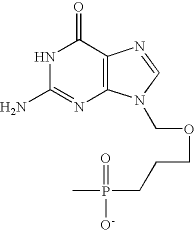 |
| HDP-phospho- PPM-DAP | 1 | CH₃(CH₂)₁₅O | H | CH₂ | 1 | H | H | absent | 0 | 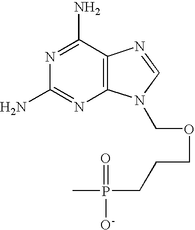 |
| HDP-phospho- PPM-N⁶cPr- DAP | 1 | CH₃(CH₂)₁₅O | H | CH₂ | 1 | H | H | absent | 0 | 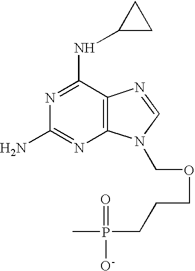 |
| HDP-phospho- PME-5FU | 1 | CH₃(CH₂)₁₅O | H | CH₂ | 1 | H | H | absent | 0 | 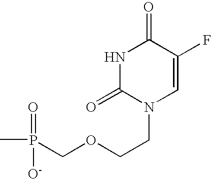 |
| HDP-phospho- PME-5FC | 1 | CH₃(CH₂)₁₅O | H | CH₂ | 1 | H | H | absent | 0 | 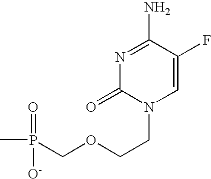 |
| HDP-phospho- HPMP-5FC | 1 | CH₃(CH₂)₁₅O | H | CH₂ | 1 | H | H | absent | 0 | 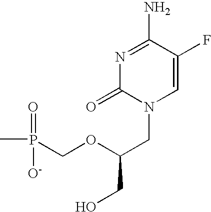 |
| HDP-phospho- HPMP-5FU | 1 | CH₃(CH₂)₁₅O | H | CH₂ | 1 | H | H | absent | 0 | 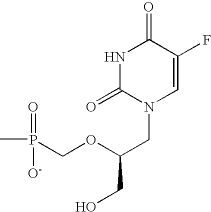 |
| HDP-phospho- Phosphonomethoxy- 3TC | 1 | CH₃(CH₂)₁₅O | H | CH₂ | 1 | H | H | absent | 0 | 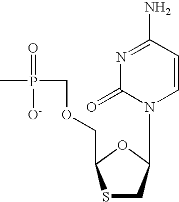 |
| HDP-phospho- Phosphonomethoxy- 2′-C-methyl ribo- guanine | 1 | CH₃(CH₂)₁₅O | H | CH₂ | 1 | H | H | absent | 0 | 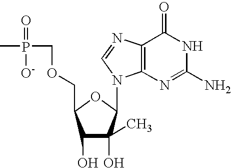 |
| HDP-phospho- Phosphonomethoxy- 1′-methyl cytidine | 1 | CH₃(CH₂)₁₅O | H | CH₂ | 1 | H | H | absent | 0 | 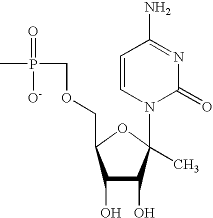 |
| HDP-phospho- PM-2′-O-methyl cytidine | 1 | CH₃(CH₂)₁₅O | H | CH₂ | 1 | H | H | absent | 0 | 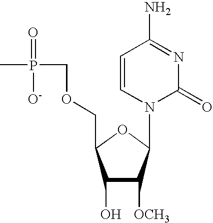 |
| ODE-phospho- PM-2′-C-methyl adenosine | 1 | CH₃(CH₂)₁₇O | H | absent | 0 | H | H | absent | 0 | 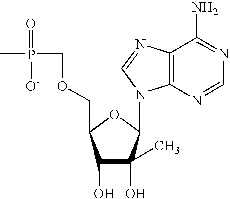 |

In certain embodiments, the compound is selected from

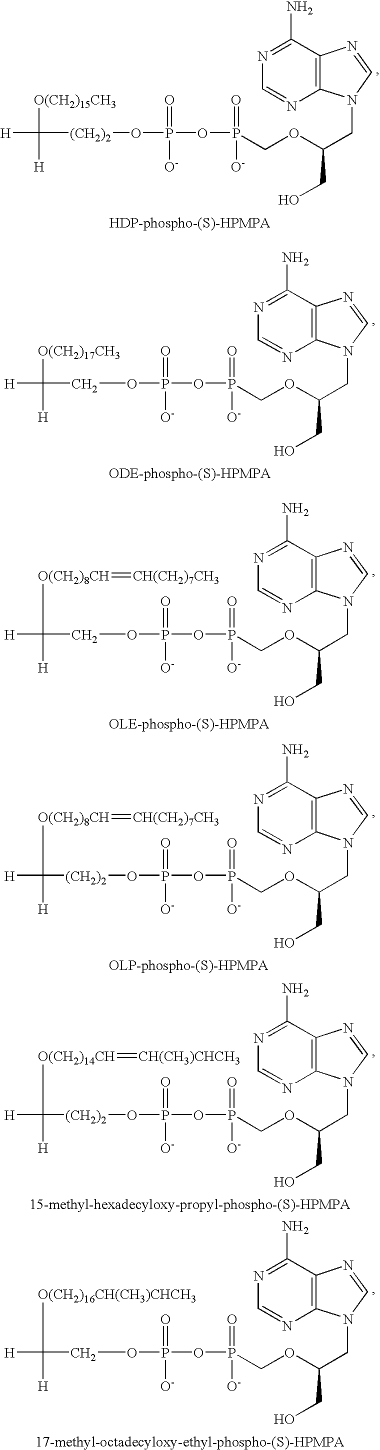

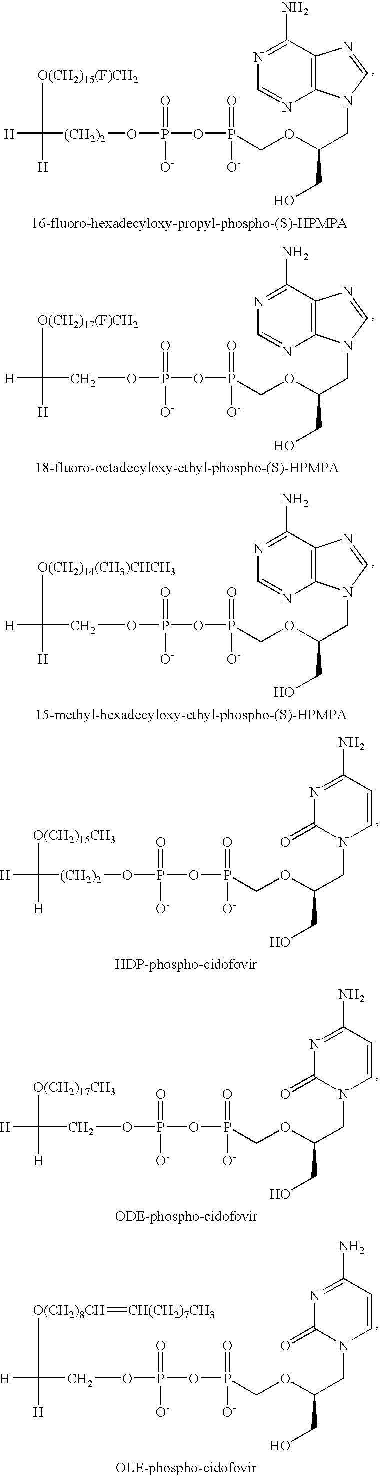

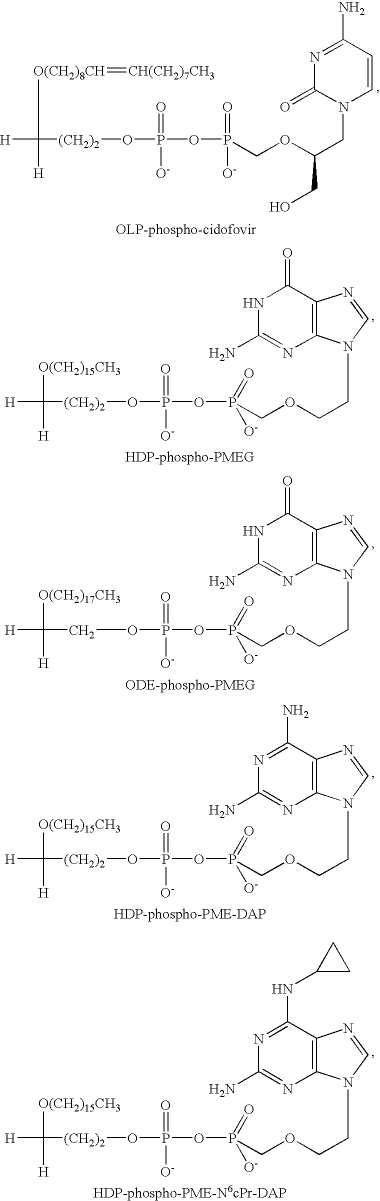

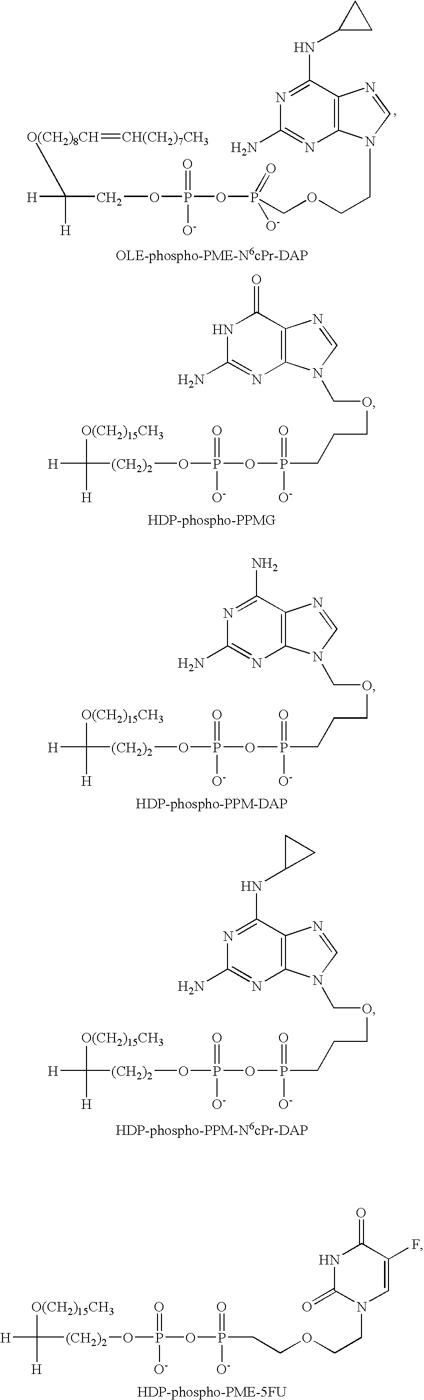

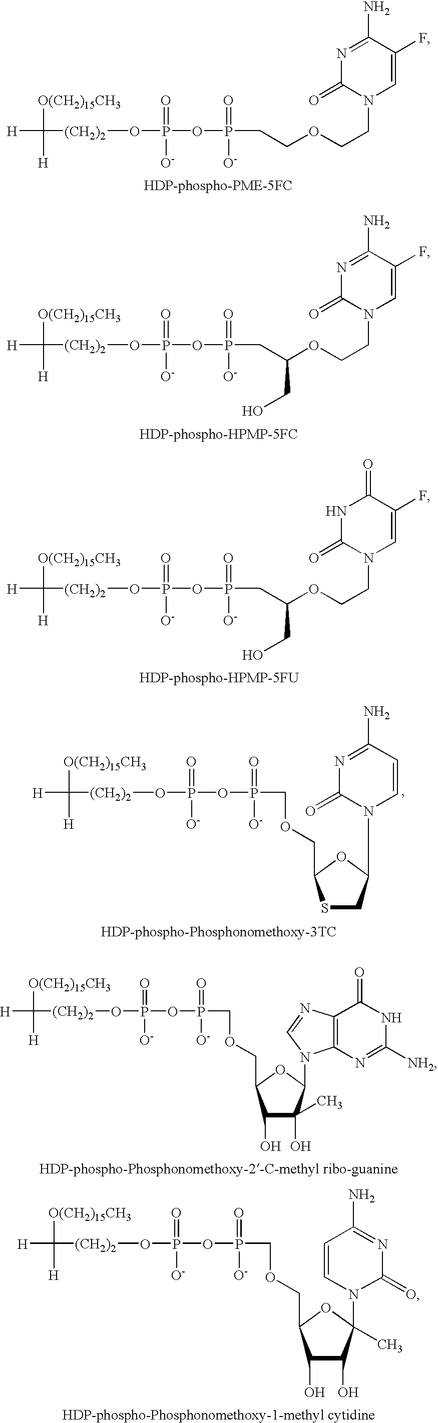

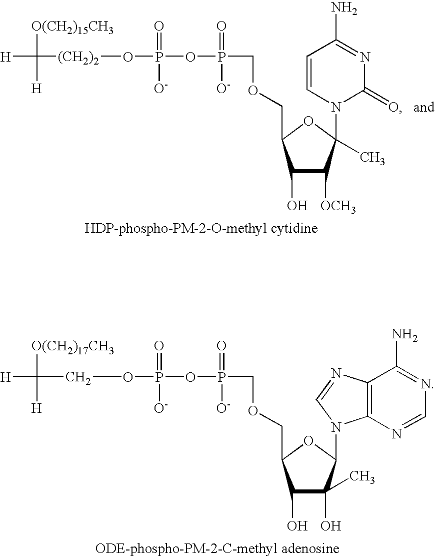

### C. Preparation of the Compounds

Exemplary methods for the preparation of nucleoside phosphonate-phosphate ester conjugates provided herein are depicted in Schemes 1 and 2. Scheme 1 outlines the synthesis of alkoxyalkyl phospho-morpholidates 3 and 4. Detailed synthesis is described in detail in example 1. In scheme 2, (S)-1-(3-hydroxy-2-phosphonomethoxypropyl)cytosine (HPMPC) is treated with dimethoxytritylchloride in DMSO by the method of Otmar et. al., An Alternative Synthesis of HPMPC and HPMPA diphosphoryl derivatives, Collection Symposium Series 2 (Chemistry of Nucleic Acid Components), 252-54, 1999, to give the intermediate 5 that is condensed with hexadecyloxypropyl-phosphate morpholidate (3) or octadecyloxyethyl-phosphate morpholidate (4) in pyridine, tributylamine and catalytic acetic acid at room temperature. Finally, hydrolysis with TFA in CHCl₃ gives compounds 6, hexadecyloxypropyl-phospho-cidofovir (HDP-phospho-HPMPC), and 7, octadecyloxyethyl-phospho-cidofovir (ODE-phospho-HPMPC).

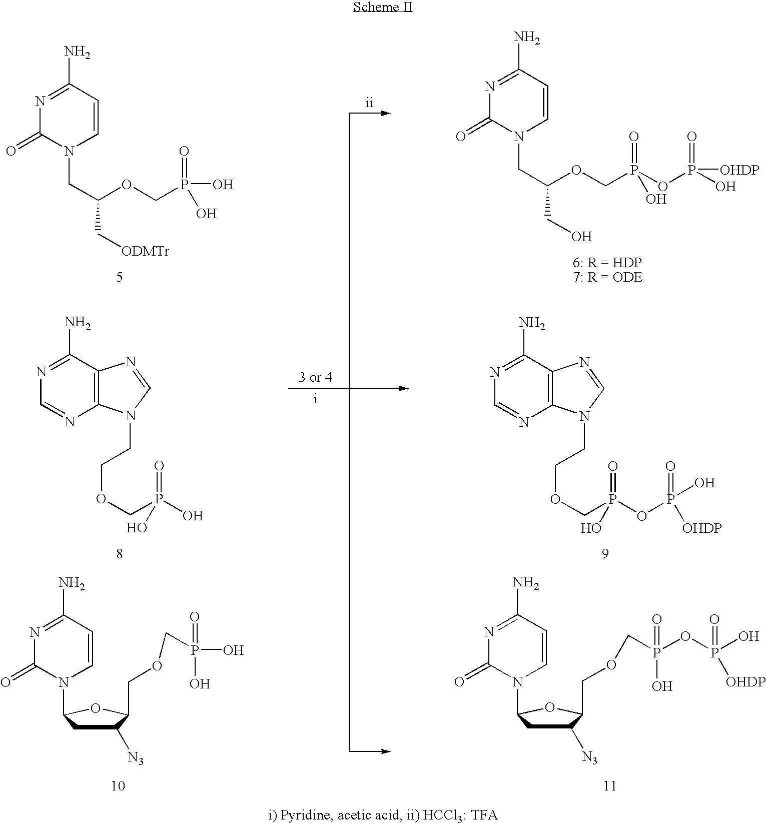

Compound 9 is prepared from the condensation of compound 89-(2-phosphonylmethoxyethyl)adenine (PMEA) and compound 3 in pyridine and acetic acid as catalyst. The synthesis of compound II is achieved by the reaction between 3′-azido-3′-deoxythymidine and diethyl[p-toluenesulfonyl-oxy]methylphosphonate in the presence of NaH, followed by hydrolysis with TMSBr to obtain the phosphonate intermediate 10 which is finally reacted with 3 to give the hexadecyloxypropyl-phospho conjugate 1.

### D. Formulation of Pharmaceutical Compositions

The pharmaceutical compositions provided herein contain therapeutically effective amounts of one or more of the compounds provided herein that are useful in the prevention, treatment, or amelioration of one or more of the symptoms of diseases or disorders associated with viral infections and inappropriate cell proliferation and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers.

In one embodiment, the compounds described above are formulated into pharmaceutical compositions using techniques and procedures well known in the art ((see, e.g., Ansel Introduction to Pharmaceutical Dosage Forms, Seventh Edition 1999).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with associated with viral infections or inappropriate cell proliferation. In one embodiment, the compositions are formulated for single dosage administration. To formulate a composition, the weight fraction of a compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems well known to those of skill in the art and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with viral infections or inappropriate cell proliferation, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refer to physically discrete units suitable for human and animal subjects and packaged individually as is known in the art.

Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In certain embodiments, the compositions are lactose-free compositions containing excipients that are well known in the art and are listed, for example, in the U.S. Pharmacopeia (USP) 25-NF20 (2002). In general, lactose-free compositions contains active ingredients, a binder/filler, and a lubricant in pharmaceutically compatible and pharmaceutically acceptable amounts. Particular lactose-free dosage forms contain active ingredients, microcrystalline cellulose, pre-gelatinized starch, and magnesium stearate.

Further provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. No. RE28,819 and U.S. Pat. No. 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids.

The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Sustained Release Dosage Form

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multi layer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

4. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some embodiments, the excipient is selected from dextrose, sorbital, fructose, corn syrup, xylitol, glycerin, glucose, sucrose and other suitable agent. The solvent may contain a buffer, such as citrate, sodium or potassium phosphate or other such buffer known to those of skill in the art at, at about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

5. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

For nasal administration, the preparation may contain an esterified phosphonate compound dissolved or suspended in a liquid carrier, in particular, an aqueous carrier, for aerosol application. The carrier may contain solubilizing agents such as propylene glycol, surfactants, absorption enhancers such as lecithin or cyclodextrin, or preservatives.

These solutions, particularly those intended for ophthalmic use, may be formulated as 0.01%-10% isotonic solutions, pH about 5-7, with appropriate salts.

6. Compositions for Other Routes of Administration

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm. Tablets and capsules for rectal administration are manufactured using the same pharmaceutically acceptable substance and by the same methods as for formulations for oral administration.

7. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLVs) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

8. Articles of Manufacture

The compounds or pharmaceutically acceptable derivatives may be packaged as articles of manufacture containing packaging material, a compound or pharmaceutically acceptable derivative thereof provided herein, which is effective for treatment, prevention or amelioration of one or more symptoms of diseases or disorders associated with viral infections or inappropriate cell proliferation, within the packaging material, and a label that indicates that the compound or composition, or pharmaceutically acceptable derivative thereof, is used for the treatment, prevention or amelioration of one or more symptoms of diseases or disorders associated with viral infections or inappropriate cell proliferation.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder associated with viral infections or inappropriate cell proliferation.

### E. Dosages

In human therapeutics, the physician will determine the dosage regimen that is most appropriate according to a preventive or curative treatment and according to the age, weight, stage of the disease and other factors specific to the subject to be treated. The pharmaceutical compositions, in another embodiment, should provide a dosage of from about 0.001 mg to about 2000 mg of compound per kilogram of body weight per day. Pharmaceutical dosage unit forms are prepared, e.g., to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The amount of active ingredient in the formulations provided herein, which will be effective in the prevention or treatment of a disorder or one or more symptoms thereof, will vary with the nature and severity of the disease or condition, and the route by which the active ingredient is administered, The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject or sample weight (e.g., from about 1 micrograms per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 110 days, 15 days, 30 days, 45 days, 2 months, 75 days, 3 months, or 6 months.

### F. Evaluation of the Activity of the Compounds

The activity of the compounds as antivirals can be measured in standard assays known in the art. Exemplary assays include, but are not limited to, plaque reduction assay in HFF cells, DNA reduction assay in MRC-5 cells, p24 reduction assay in MT-2 cells, CPE assay in HFF cells and EBV Elisa assay in Daudi cells.

In Table 3, EC₅₀ for the compounds provided herein and tested against various viruses in vitro are provided.

### G. Methods of Use of the Compounds and Compositions

Methods of treating, preventing, or ameliorating one or more symptoms of diseases associated with viral infections or inappropriate cell proliferation using the compounds and compositions are provided. In practicing the methods, effective amounts of the compounds or compositions containing therapeutically effective concentrations of the compounds are administered. In certain embodiments, the methods provided herein are for the preventing, or ameliorating one or more symptoms of diseases associated with viral infections, including, but not limited to influenza; hepatitis B and C virus; cytomegalovirus (CMV); herpes infections, such as those caused by Varicella zoster virus, Herpes simplex virus types 1 & 2, Epstein-Barr virus, Herpes type 6 (HHV-6) and type 8 (HHV-8); Varicella zoster virus infections such as shingles or chicken pox; Epstein Barr virus infections, including, but not limited to infectious mononucleosis/glandular; retroviral infections including, but not limited to SIV, HIV-1 and HIV-2; ebola virus; adenovirus and papilloma virus.

In further embodiments, the methods provided herein are for treating, preventing, treating, or ameliorating one or more symptoms of diseases associated with viral infections caused by orthopox viruses, such as variola major and minor, vaccinia, smallpox, cowpox, camelpox, and monkeypox. In certain embodiments, the disease is drug resistant hepatitis B.

In certain embodiments, the methods provided herein are for treating, preventing, or ameliorating one or more symptoms of diseases associated with cell proliferation, including, but not limited to cancers. Examples of cancers include, but are not limited to, lung cancer, head and neck squamous cancers, colorectal cancer, prostate cancer, breast cancer, acute lymphocytic leukemia, adult acute myeloid leukemia, adult non Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, and skin cancer.

### H. Combination Therapy

The compounds and compositions provided herein may also be used in combination with one or more other active ingredients. In certain embodiments, the compounds may be administered in combination, or sequentially, with another therapeutic agent. Such other therapeutic agents include those known for treatment, prevention, or amelioration of one or more symptoms associated with viral infections or inappropriate cell proliferation. Such therapeutic agents include, but are not limited to, antiviral agents and anti-neoplastic agents.

Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameter of the drug can be altered by such combination or alternation therapy.

In certain embodiments, provided herein are methods of treatment, prevention or amelioration that encompass administration of a second agent effective for the treatment, prevention or amelioration of viral infection, such as HIV and/or HCV infection. The second agent can be any agent known to those of skill in the art to be effective for the treatment, prevention or amelioration of viral infections, such as the HIV and/or HCV infection. The second agent can be a second agent presently known to those of skill in the art, or the second agent can be second agent later developed for the treatment, prevention or amelioration of viral infections. In certain embodiments, the second agent is presently approved for the treatment or prevention of HIV and/or HCV.

In certain embodiments, a compound provided herein is administered in combination with one second agent. In further embodiments, a second agent is administered in combination with two second agents. In still further embodiments, a second agent is administered in combination with two or more second agents.

In one embodiment, the second antiviral agent for the treatment of HIV can be a reverse transcriptase inhibitor (a “RTI”), which can be either a synthetic nucleoside (a “NRTI”) or a non-nucleoside compound (a “NNRTI”). In an alternative embodiment, in the case of HIV, the second (or third) antiviral agent can be a protease inhibitor. In other embodiments, the second (or third) compound can be a pyrophosphate analog, or a fusion binding inhibitor.

In some embodiments, compounds for combination or alternation therapy for the treatment of HBV include, but are not limited to 3TC, FTC, L-FMAU, interferon, β-D-dioxolanyl-guanine (DXG), β-D-dioxolanyl-2,6-diaminopurine (DAPD), and β-D-dioxolanyl-6-chloropurine (ACP), famciclovir, penciclovir, BMS-200475, bis pom PMEA (adefovir, dipivoxil); lobucavir, ganciclovir, and ribavarin.

In another embodiment, examples of antiviral agents that can be used in combination or alternation with the compounds disclosed herein for HIV therapy include cis-2-hydroxymethyl-5-(5-fluorocytosin-1-yl)-1,3-oxathiolane (FTC); the (-;)-enantiomer of 2-hydroxymethyl-5-(cytosin-1-yl)-1,3-oxathiolane (3TC); carbovir, acyclovir, foscamet, interferon, AZT, DDI, DDC, D4T, CS-87 (3′-azido-2′,3′-dideoxy-uridine), and β-D-dioxolane nucleosides such as β-D-dioxolanyl-guanine (DXG), β-D-dioxolanyl-2,6-diaminopurine (DAPD), and β-D-dioxolanyl-6-chloropurine (ACP), MKC442 (6-benzyl-1-(ethoxymethyl)-5-isopropyl uracil.

The protease inhibitors include crixivan, nelfinavir, ritonavir, saquinavir, DMP-266 and DMP-450.

Further compounds that can be administered in combination or alternation with any of the compounds provided herein include (1S,4R)-4-[2-amino-6-cyclopropyl-amino)-9H-purin-9-yl]-2-cyclopentene-1-methanol succinate (“1592”, a carbovir analog); 3TC; -β-L-2′,3′-dideoxy-3′-thiacytidine; a-APA R18893: a-nitro-anilino-phenylacetamide; A-77003; C2 symmetry-based protease inhibitor; A-75925: C2 symmetry-based protease inhibitor; AAP-BHAP: bisheteroarylpiperazine analog; ABT-538: C2 symmetry-based protease inhibitor; AzddU: 3′-azido-2′,3′-dideoxyuridine; AZT: 3′-azido-3′-deoxythymidine; AZT-p-ddI: 3′-azido-3′-deoxythymidilyl-(5′,5′)-2′,3′-dideoxyinosinic acid; BHAP: bisheteroarylpiperazine; BILA 1906: N-{1S-[[[3-[2S-{(1,1-dimethylethyl)amino]carbonyl}-4R-]3-pyridinylmethyl)thio]-1-piperidinyl]-2R-hydroxy-1S-(phenylmethyl)propyl]amino]carbonyl]-2-methylpropyl}-2-quinolinecarboxamide; BILA 2185: N-(1,1-dimethylethyl)-1-[2S-[[2-2,6-dimethyphenoxy)-1-oxoethyl]amino]-2R-hydroxy-4-phenylbutyl]-4R-pyridinylthio)-2-piperidine-carboxamide; BM+51.0836: thiazolo-isoindolinone derivative; BMS 186,318: aminodiol derivative HIV-1 protease inhibitor; d4API: 9-[2,5-dihydro-5-(phosphonomethoxy)-2-furanyl]adenine; d4C: 2′,3′-didehydro-2′,3′-dideoxycytidine; d4T: 2′,3′-didehydro-3′-deoxythymidine; ddC; 2′,3′-dideoxycytidine; ddI: 2′,3′-dideoxyinosine; DMP-266: a 1,4-dihydro-2H-3,1-benzoxazin-2-one; DMP-450: {[4R-(4-a,5-a,6-b,7-b)]-hexahydro-5,6-bis(hydroxy)-1,3-bis(3-amino)phenyl]methyl)-4,7-bis(phenylmethyl)-2H-1,3-diazepin-2-one}-bismesylate; DXG: (-;)-β-D-dioxolane-guanosine; EBU-dM: 5-ethyl-1-ethoxymethyl-6-(3,5-dimethylbenzyl)uracil; E-EBU: 5-ethyl-1-ethoxymethyl-6-benzyluracil; DS: dextran sulfate; E-EPSeU: 1-(ethoxymethyl)-(6-phenylselenyl)-5-ethyluracil; E-EPU: 1-(ethoxymethyl)-(6-phenyl-thio)-5-ethyluracil; FTC: β-2′,3′-dideoxy-5-fluoro-3′-thiacytidine (Triangle); HBY097: S-4-isopropoxycarbonyl-6-methoxy-3-(methylthio-methyl)-3,4-dihydroquinoxalin-2(1H)-thione; HEPT: 1-[(2-hydroxyethoxy)methyl]-6-(phenylthio)thymine; HIV-1: human immunodeficiency virus type 1; JM2763: 1,1′-(1,3-propanediyl)-bis-1,4,8,11-tetraazacyclotetradecane; JM3100: 1,1′-[1,4-phenylenebis-(methylene)]-bis-1,4,8,11-tetraazacyclotetradecane; KNI-272: (2S,3S)-3-amino-2-hydroxy-4-phenylbutyric acid-containing tripeptide; L-697,593; 5-ethyl-6-methyl-3-(2-phthalimido-ethyl)pyridin-2(1H)-one; L-735,524: hydroxy-aminopentane amide HIV-1 protease inhibitor; L-697,661: 3-{[(-4,7-dichloro-1,3-benzoxazol-2-yl)methyl]amino}-5-ethyl-6-methylpyridin-2(1H)-one; L-FDDC: (-;)-β-L-5-fluoro-2′,3′-dideoxycytidine; L-FDOC: (-;)-β-L-5-fluoro-dioxolane cytosine; MKC442: 6-benzyl-1-ethoxymethyl-5-isopropyluracil (I-EBU); Nevirapine: 11-cyclopropyl-5,11-dihydro-4-methyl-6H-dipyridol[3,2-b: 2′,3′-e]diazepin-6-one; NSC648400: 1-benzyloxymethyl-5-ethyl-6-(alpha-pyridylthio)uracil (E-BPTU); P9941: [2-pyridylacetyl-IlePheAla-y(CHOH)]2; PFA: phosphonoformate; PMEA: 9-(2-phosphonylmethoxyethyl)adenine; PMPA: (R)-9-(2-phosphonyl-methoxypropyl)adenine; Ro 31-8959: hydroxyethylamine derivative HIV-1 protease inhibitor; RPI-312: peptidyl protease inhibitor, 1-[(3s)-3-(n-alpha-benzyloxycarbonyl)-1-asparginyl)-amino-2-hydroxy-4-phenylbutyryl]-n-tert-butyl-1-proline amide; 2720: 6-chloro-3,3-dimethyl-4-(isopropenyloxycarbonyl)-3,4-dihydro-quinoxalin-2(1H)thione; SC-52151: hydroxyethylurea isostere protease inhibitor; SC-55389A: hydroxyethyl-urea isostere protease inhibitor; TIBO R82150: (+)-(5S)-4,5,6,7-tetrahydro-5-methyl-6-(3-methyl-2-butenyl)imidazo[4,5,1-jk][1,4]-benzodiazepin-2(1H)-thione; TIBO 82913: (+)-(SS)-4,5,6,7,-tetrahydro-9-chloro-5-methyl-6-(3-methyl-2-butenyl)imidazo[4,5,1jk]-[1,4]benzo-diazepin-2(1H)-thione; TSAO-m3T: [2′,5′-bis-O-(tert-butyldimethylsilyl)-3′-spiro-5′-(4′-amino-1′,2′-oxathiole-2′,2′-dioxide)]-b-D-pentofiaranosyl-N-3-methylthymine; U90152: 1-[3-[(1-methylethyl)-amino]-2-pyridinyl]-4-[[5-[(methylsulphonyl)-amino]-1H-indol-2-yl]carbonyl]-piperazine; UC: thiocarboxanilide derivatives (Uniroyal); UC-781=N-[4-chloro-3-(3-methyl-2-butenyloxy)phenyl]-2-methyl-3-furancarbothioamide; UC-82=N-[4-chloro-3-(3-methyl-2-butenyloxy)phenyl]-2-methyl-3-thiophenecarbothioamide; VB 11,328: hydroxyethyl-sulphonamide protease inhibitor; VX-478: hydroxyethylsulphonamide protease inhibitor; XM 323: cyclic urea protease inhibitor.

In certain embodiments, suitable second agents include small-molecule, orally bioavailable inhibitors of the HCV enzymes, nucleic-acid-based agents that attack viral RNA, agents that can modulate the host immune response. Exemplary second agents include: (i) current approved therapies (peg-interferon plus ribavirin), (ii) HCV-enzyme targeted compounds, (iii) viral-genome-targeted therapies (e.g., RNA interference or RNAi), and (iv) immunomodulatory agents such as ribavirin, interferon (INF) and Toll-receptor agonists.

In certain embodiments, the second agent is a modulator of the NS3-4A protease. The NS3-4A protease is a heterodimeric protease, comprising the amino-terminal domain of the NS3 protein and the small NS4A cofactor. Its activity is essential for the generation of components of the viral RNA replication complex.

One useful NS34A protease inhibitor is BILN 2061 (Ciluprevir; Boehringer Ingelheim), a macrocyclic mimic of peptide product inhibitors. Although clinical trials with BILN 2061 were halted (preclinical cardiotoxicity), it was the first NS3 inhibitor to be tested in humans. See Lamarre et al., 2003, Nature 426:186-189, the contents of which are hereby incorporated by reference in their entirety.

Another useful NS3-4A protease inhibitor is VX-950 (Vertex/Mitsubishi), a protease-cleavage-product-derived peptidomimetic inhibitor of the NS3-4A protease. It is believed to be stabilized into the enzyme's active site through a ketoamide. See, e.g., Lin et al., 2005, J Biol Chem. Manuscript M506462200 (epublication); Summa, 2005, Curr Opin Investig Drugs. 6:831-7, the contents of which are hereby incorporated by reference in their entireties.

In certain embodiments, the second agent is a modulator of the HCV NS5B The RNA-dependent RNA polymerase (RdRp). Contained within the NS5B protein, RdRp synthesizes RNA using an RNA template. This biochemical activity is not present in mammalian cells.

One useful modulator of RdRp is NM283 (Valopicitabine; Idenix/Novartis). NM283, is an oral prodrug (valine ester) of NM107 (2-C-methyl-cytidine) in phase II trials for the treatment or prevention of HCV infection. See, e.g., U.S. Patent Application Publication No. 20040077587, the contents of which are hereby incorporated by reference in their entirety.

Other useful modulators of RdRp include 7-deaza nucleoside analogs. For instance, 7-Deaza-2′-C-methyl-adenosine is a potent and selective inhibitor of hepatitis C virus replication with excellent pharmacokinetic properties. Olsen et al., 2004, Antimicrob. Agents Chemother. 48:3944-3953, the contents of which are hereby incorporated by reference in their entirety.

In further embodiments, the second agent is a non-nucleoside modulator of NS5B. At least three different classes of non-nucleoside inhibitors (NNI) of NS5B inhibitors are being evaluated in the clinic.

Useful non-nucleoside modulators of NS5B include JTK-003 and JTK-009. JTK-003 has been advanced to phase II. Useful non-nucleoside modulators of NS5B include the 6,5-fused heterocyclic compounds based on a benzimidazole or indole core. See, e.g., Hashimoto et al., WO 00147883, the contents of which are hereby incorporated by reference in their entirety.

Further useful polymerase NNIs include R803 (Rigel) and HCV-371, HCV-086 and HCV-796 (ViroPharma/Wyeth). Additional useful NNIs include thiophene derivatives that are reversible allosteric inhibitors of the NS5B polymerase and bind to a site that is close to, but distinct from, the site occupied by benzimidazole-based inhibitors. See, e.g., Biswal, et al., 2005, J Biol. Chem. 280, 18202-18210 (2005).

Further useful NNIs for the methods provided herein include benzothiadiazines, such as benzo-1,2,4-thiadiazines. Derivatives of benzo-1,2,4-thiadiazine have been shown to be highly selective inhibitors of the HCV RNA polymerase. Dhanak, et al., 2002, J. Biol. Chem. 277:38322-38327, the contents of which are hereby incorporated by reference in their entirety.

Further useful NNIs for the methods provided herein, and their mechanisms, are described in LaPlante et al. 2004 Angew Chem. Int. Ed. Engl. 43:4306-4311; Tomei et al., 2003, J. Virol. 77:13225-13231; Di Marco et al., 2005, J. Biol. Chem. 280:29765-70; Lu, H., WO 2005/000308; Chan et al., 2004, Bioorg. Med. Chem. Lett. 14:797-800; Chan et al., 2004, Bioorg. Med. Chem. Lett. 14:793-796; Wang et al., 2003, J. Biol. Chem. 278:9489-9495; Love, et al., 2003, J. Virol. 77:7575-7581; Gu et al., 2003, J. Biol. Chem. 278:16602-16607; Tomei et al., 2004, J. Virol. 78:938-946; and Nguyen et al, 2003, Antimicrob. Agents Chemother. 47:3525-3530; the contents of each are hereby incorporated by reference in their entireties.

In a further embodiment, the second agent is an agent that is capable of interfering with HCV RNA such as small inhibitory RNA (siRNA) or a short hairpin RNA (shRNA) directed to an HCV polynucleotide. In tissue culture, siRNA and vector-encoded short hairpin RNA shRNA directed against the viral genome, effectively block the replication of HCV replicons. See, e.g., Randall et al., 2003, Proc. Natl Acad. Sci. USA 100:235-240, the contents of which are hereby incorporated by reference in their entirety.

In a further embodiment, the second agent is an agent that modulates the subject's immune response. For instance, in certain embodiments, the second agent can be a presently approved therapy for HCV infection such as an interferon (IFN), a pegylated IFN, an IFN plus ribavirin or a pegylated IFN plus ribavirin. In certain embodiments, interferons include IFNα, IFNα2a and IFNα2b, and particularly pegylated IFNα2a (PEGASYS®) or pegylated IFNα2b (PEG-INTRON®).

In a further embodiment, the second agent is a modulator of a Toll-like receptor (TLR). It is believed that TLRs are targets for stimulating innate anti-viral response. Suitable TLRs include, bur are not limited to, TLR3, TLR7, TLR8 and TLR9. It is believed that toll-like receptors sense the presence of invading microorganisms such as bacteria, viruses and parasites. They are expressed by immune cells, including macrophages, monocytes, dendritic cells and B cells. Stimulation or activation of TLRs can initiate acute inflammatory responses by induction of antimicrobial genes and pro-inflammatory cytokines and chemokines.

In certain embodiments, the second agent is a polynucleotide comprising a CpG motif. Synthetic oligonucleotides containing unmethylated CpG motifs are potent agonists of TLR-9. Stimulation of dendritic cells with these oligonucleotides results in the production of tumour necrosis factor-alpha, interleukin-12 and IFN-alpha TLR-9 ligands are also potent stimulators of B-cell proliferation and antibody secretion. One useful CpG-containing oligonucleotide is CPG-10101 (Actilon; Coley Pharmaceutical Group) which has been evaluated in the clinic.

Another useful modulator of a TLR is ANA975 (Anadys). ANA975 is believed to act through TLR-7, and is known to elicit a powerful anti-viral response via induction and the release of inflammatory cytokines such as IFN-alpha.

In another embodiment, the second agent is Celgosivir. Celgosivir is an alpha-glucosidase inhibitor and acts through host-directed glycosylation. In preclinical studies, celgosivir has demonstrated strong synergy with IFNα plus ribavirin. See, e.g., Whitby et al., 2004, Antivir Chem. Chemother. 15(3):141-51. Celgosivir is currently being evaluated in a Phase II monotherapy study in chronic HCV patients in Canada.

Further immunomodulatory agents, and their mechanisms or targets, are described in Schetter & Vollmer, 2004, Curr. Opin. Drug Discov. Dev. 7:204-210; Takeda et al., 2003, Annu. Rev. Immunol. 21:335-376; Lee et al., 2003, Proc. Natl. Acad. Sci. USA 100:6646-6651; Hosmans et al., 2004, Hepatology 40 (Suppl. 1), 282A; and U.S. Pat. No. 6,924,271; the contents of each are hereby incorporated by reference in their entireties.

In certain embodiments, the compounds provided herein may be administered in combination with one or more anti-cancer agents. Anti-cancer agents for use in combination with the instant compounds include, but are not limited to, an antifolate, a 5-fluoropyrimidine (including 5-fluorouracil), a cytidine analogue such as 13-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine, an alkylating agent (including but not limited to busulfan, chlorambucil, cyclophosphamide, ifofamide, mechlorethamine, melphalan, and thiotepa), nonclassical alkylating agents, platinum containing compounds, bleomycin, an anti-tumor antibiotic, an anthracycline such as doxorubicin and dannomycin, an anthracenedione, topoisomerase H inhibitors, hormonal agents (including but not limited to corticosteroids (dexamethasone, prednisone, and methylprednisone), androgens such as fluoxymesterone and methyltestosterone, estrogens such as diethylstilbesterol, antiestrogens such as—tamoxifen, LHRH analogues such as leuprolide, antiandrogens such as flutamide, aminoglutethimide, megestrol acetate, and medroxyprogesterone), asparaginase, carmustine, lomustine, hexamethyl-melamine, dacarbazine, mitotane, streptozocin, cisplatin, carboplatin, levamasole, and leucovorin. The compounds provided herein can also be used in combination with enzyme therapy agents and immune system modulators such as an interferon, interleukin, tumor necrosis factor, macrophage colony-stimulating factor and colony stimulating factor.

It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients.

The following examples are provided for illustrative purposes only and are not intended to limit the scope of the invention.

### EXAMPLES

### Example 1

### 2-(octadecyloxy)ethyl dihydrogen phosphate (2)

To a cold solution of phosphorus oxychloride, 3 ml (32 mmol) in THF was added dropwise a solution of 2-octadecyloxy-1-ethanol (5 g, 16 mmol) and triethylamine (4.4 ml, 32 mmol) in THF, while the temperature was maintained below 20° C. After addition was complete, the mixture was kept an additional hour, then water was added and the stirring was continued overnight. The mixture was then extracted with ethyl ether and the ether layer was concentrated. The crude solid was recrystallized from hexane to give octadecyloxyethyl phosphate as a white solid in 72% yield.

### 2-(octadecyloxy)ethyl hydrogen morpholinophosphate (4)

To a solution of 3 g (7.6 mmol) of 2-(octadecyloxy)ethyl dihydrogen phosphate in tert-butyl alcohol was added 2 g (24 mmol) of morpholine and 5.8 g (30 mmol) of DCC, added in four portions and refluxed over 48 h. Ethyl ether was added to the mixture, then it was filtered, and the filtrate concentrated to give 2-(octadecyloxy)ethyl phosphonomorpholidate as an oil. Mass spectrum: (ESI) m/z 462 (M−H)⁻.

### Example 2

### 3-(hexadecyloxy)propyl dihydrogen phosphate (1)

1 was prepared following the same procedure described in example 1, except that 3-(hexadecyloxy)-1-propanol was phosphorylated.

### 3-(hexadecyloxy)propyl hydrogen mopholinophosphate (3)

HDP-phospho-morpholidate was prepared using the procedure described in example 1 using 1.92 g (15 mmol) of 3-(hexadecyloxypropyl) dihydrogen phosphate, 1.3 g (15 mmol) of morpholine and 35 mmol of DCC. HDP-phospho-morpholidate was obtained as an oil. Mass spectrum: (ESI) m/z 449 (M−H)⁻.

### Example 3

### (S)-1-(3-(4,4′-dimethoxytrityloxy)-2-phosphonomethoxypropyl)cytosine (5)

(S)-1-(3-hydroxy-2-phosphonomethoxypropyl)cytosine dihydrate (free acid, 0.25 g, 0.75 mmol) was mixed with methanol and 3 ml of tributylamine. After solution was achieved, solvents were removed to dryness and the solid residue was dissolved in DMSO. To this solution was added 0.9 g of tributylamine and 0.7 g of dimethoxytrityl chloride, and the mixture was stirred overnight at room temperature. Solvents were removed and the solid residue was recrystallized from ethyl acetate and dried under vacuum to obtain 0.35 g (91%) of the product 5 as a white solid. MS (ESI) m/z 943 (M−H)⁻.

### Example 4

### (1-(4-aminooxopyrimidin-1(2H)-yl)-3-hydroxypropan-2-yloxy)methylphosphonic(3-hexadecyloxy)propylphosphoric) anhydride (HDP-phospho-cidofovir, 6)

To a suspension of (5) 0.1 g (0.17 mmol) in pyridine was added 0.1 ml of tributylamine. After solution was achieved, a solution of 1 g of 3 in pyridine was added along with 0.1 ml of acetic acid. The reaction was stirred at room temperature for 48 h. Solvents were removed to dryness and the residue was mixed with a solution of CHCl₃ and TFA (5:0.5) and stirred at room temperature. Solvents were removed and the crude mixture was purified by flash column chromatography using silica gel and eluting with 70:30:3:3 (CHCl₃:Methanol:Ammonium hydroxide:Water) to obtain 0.04 g (40%) of 6 as a white solid. MS (EST) m/z 642 (M−H)⁻. ¹H NMR 300 MHz (DMSO) δ ppm: 7.47 (d, 1H, 6.9 Hz), 5.66 (d, 1H, 7.2 Hz), 3.8-3.1 (m, 16H), 1.7 (m, 2H), 1.4 (m, 2H), 1.2 (s, 26H), 0.8 (t, 3H). ³¹P NMR (DMSO-d₆) 12.5 (s, P1), 0.4 (s, P2).

### Example 5

### (1-(4-aminooxopyrimidin-1(2H)-yl)-3-hydroxypropan-2-yloxy)methylphosphonic-(3-octadecyloxy)ethyl phosphoric) anhydride (ODE-phospho-cidofovir, 7)

To a suspension of 5 (0.14 g, 0.17 mmol) in pyridine was added 0.1 ml of tributylamine. After solution was achieved, a solution of 1 g of 4 in pyridine was added along with 0.1 ml of acetic acid. The reaction was stirred at room temperature for 48 h. Solvents were removed to dryness and the residue was mixed with a solution of CHCl₃:TFA (5:0.5) and stirred at room temperature. After complete deprotection the crude mixture was purified by column chromatography using silica gel and eluting with 70:30:3:3 (CHCl₃:Methanol:Ammonium hydroxide:Water) to obtain 0.03 g (20%) of 7 as a white solid. MS (ESI) m/z 654 (M−H)⁻. ¹H NMR 300 MHz (CDCl₃/CD₃OD) δ ppm: 6 (d, 1H, J=8 Hz), 5.8 (d, 1H, J=7.2 Hz), 4.2-3.4 (m, 16H), 1.7 (m, 2H), 1.58 (m, 2H), 1.27 (s, 28H), 0.89 (t, 3H). ³¹P NMR (CDCl₃/CD₃OD), 12.38 (m. P1), 5.6 (d. P2, J=25.5 Hz).

### Example 6

### (2-(6amino-9H-purin-9-yl)ethoxy)methylphosphonic(3-hexadecyloxy)propylphosphonic)anhydride (HDP-phospho-PMEA, 9)

9-(2-phosphonylmethoxyethyl)adenine (PMEA) 0.5 g was dissolved in pyridine and a solution of 1 g of 3 in pyridine was added along with 1 ml of acetic acid. The reaction mixture was heated at 40° C. overnight. Solvents were removed and the oily residue was washed with 10% methanol in ethyl ether and filtered. The filtrate was concentrated and purified by column chromatography using silica gel and eluting with 25% methanol in dichloromethane to obtain 0.19 g of 9 (17%) as a white solid. Mass spectrum: (ESI) m/z 635(M−H)⁻, 636(M+H)⁺. ¹H NMR 300 MHz (DMSO) δ 8.35 (s, 1H), 8.09 (s, 1H), 7.86 (bs, 2H), 7.12 (bs, 2h), 0.83 (s, 3H). ³¹P NMR (DMSO) δ1.4 (d, P1, J_{PP}=24.32 Hz), −12 (d, P2, Jₚₚ=24.32 Hz).

### Example 7

### 5′,3′-dideoxy-5′-(oxymethylphosphonic acid)-3′-azidothymidine (10)

To a suspension of 1.2 g of NaH (60% oil dispersion) in DMF, was added a solution of 2.67 g (10 mmol) of 3′-deoxy-3′-azidothymidine (AZT). After 30 min of reaction at room temperature, a solution of 3.22 g (10 mmol) of diethyl p-toluenesulfonyloxymethylphosphonate in DMF was added. The mixture was stirred for 3 days at room temperature, then neutralized with acetic acid and the solvent was removed to dryness. The solid residue was purified by column chromatography using 5% methanol in dichloromethane to obtain 1.4 g (34%) of diethyl 5′,3′-dideoxy-5′-(oxymethylphosphonate)-3′-azidothymidine. This product was treated with TMSBr in dicholoromethane to obtain a mixture that was purified by ion exchange using DEAE in HCO₃ form, eluting with ammonium bicarbonate solution to obtain 10 as a white solid. Mass spectrum: (ESI) m/z 360 (M−H)⁻. ³¹P NMR (D₂O) 14.3 (s, P1).

### Example 8

### 5′,3′-dideoxy-5′-(oxymethylphosphonic acid)-3′-azidothymidine, (3-hexadecyloxy)propyl phosphoric anhydride (HDP-phospho-phosphonomethoxy AZT), 11)

To a solution of 0.18 g (0.49 mmol) of compound 10 in pyridine was added a solution of 1 g (2.2 mmol) of 3 in pyridine and 1 ml of acetic acid. The mixture was heated at 40° C. overnight. Solvents were removed and the residue was purified by flash column chromatography on silica gel using 70:30:3:3 (CHCl₃:Methanol:Ammonium hydroxide:Water) to obtain product 11 as a white solid. Mass spectrum: (ESI) m/z 722 (M−H)⁻. ³¹P NMR (DMSO-d₆) 5.6 (d, P1, J_{PP}=26.6 Hz), 9.5 (d, P2, J_{PP}=25.6 Hz). ¹H NMR 300 MHz (DMSO-d₆) δ 7.5 (s, 1H), 6.01 (m, 1H), 4.4-4.3 (m, 1H), 3.7-3.48 (m, 6H), 2.0-2.3 (m, 4H), 1.8-1.6 (m, 5H), 1.4 (m, 2H), 1.2 (bs, 24H), 0.83 (t, 3H).

### Example 9

### Antiviral Activity of Substituted Phosphate Esters of Nucleoside Phosphonates

Compounds provided herein were prepared and tested against various viruses in vitro. In Table 3, EC₅₀ for exemplary compounds are provided as follows:

TABLE 3

| Compound | Cowpox¹ | Vaccinia² | HCMV³ | MCMV⁴ | HSV-1⁵ | HSV-1⁶ | HSV-2⁷ | HIV-1⁸ |
|---|---|---|---|---|---|---|---|---|
| HPMPC | 42 | 31 | 1.2 | 0.04 | 3.3 | 15.0 | 10.5 | ND |
| HDP- | 3.9 | 2.8 | 0.1 | 0.35 | 0.06 | 0.13 | 0.3 | ND |
| phospho- | | | | | | | | |
| HPMPC | | | | | | | | |
| ODE- | 0.54 | 0.32 | 0.004 | 0.03 | 0.00002 | ND | ND | ND |
| phospho- | | | | | | | | |
| HPMPC | | | | | | | | |
| ADV | ND | ND | ND | ND | ND | ND | ND | 1.3 |
| HDP- | ND | ND | ND | ND | ND | ND | ND | 0.009 |
| phospho- | | | | | | | | |
| ADV | | | | | | | | |

Results expressed as 50% effective concentration, EC₅₀ in μM; Abbreviations: HDP-P-HPMPC: (1-(4-aminooxopyrimidin-1(2H)-yl)-3-hydroxypropan-2yloxy)-methylphosphonic(3-hexadecyloxy)propyl-phosphoric) anhydride (6), ODE-P-HPMPC: (1-(4-aminooxopyrimidin-1(2H)-yl)-3-hydroxypropan-2-yloxy)methylphosphonic-(3-octadecyloxy)ethyl phosphoric) anhydride, ADV: adefovir, phosphonomethoxyethyadenine; HDP-P-ADV: (2-(6amino-9H-purin-9-yl)ethoxy)methylphosphonic(3-hexadecyloxy)-propylphosphonic)anhydride; Antiviral Assays:
¹Cowpox Brighton, plaque reduction assay in HFF cells;
²Vaccinia WR plaque reduction assay in HFF cells,
³AD169 plaque reduction assay in HFF cells;
⁴Plaque reduction assay in HFF cells,
⁵HSV-1 DNA reduction assay in MRC-5 Cells,
⁶HSV-1 plaque reduction assay;
⁷HSV-2 plaque reduction assay,
⁸HIV-1_{Lai} p24 reduction assay in MT-2 cells.
ND = not determined.

Since modifications will be apparent to those of skill in the art, it is intended that the invention be limited only by the scope of the appended claims.

1. A compound of formula I: 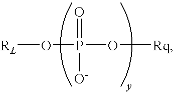
or a pharmaceutically active derivative thereof,
wherein R_{L} is a lipophilic group, R_{q} is a pharmacologically active phosphonate,
and y is 1 or 2.

2. The compound of claim 1, wherein the compound has formula II: 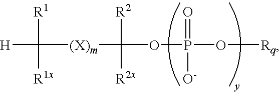
or a pharmaceutically active derivative thereof,
wherein; R¹ and R^{1x} are each independently —H, —O(C₁-C₂₄)alkyl, —O(C₂-C₂₄)alkenyl, —O(C₁-C₂₄)acyl, —S(C₁-C₂₄)alkyl, —S(C₂-C₂₄)alkenyl, or —S(C₁-C₂₄)acyl, wherein at least one of R¹ and R^{1x} is not —H, and wherein said alkenyl or acyl optionally have 1 to about 6 double bonds,
R² and R^{2x} are each independently —H, —O(C₁-C₇)alkyl, —O(C₂-C₇)alkenyl, S(C₁-C₇)alkyl, —S(C₂-C₇)alkenyl, —O(C₁-C₇)acyl, —S(C₁-C₇)acyl, —N(C₁-C₇)acyl, NH(C₁-C₇)alkyl, —N((C₁-C₇)alkyl)₂, halogen, —NH₂, —OH, or —SH; X, when present, is: 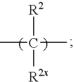
m is an integer from 0 to 6; n is or; and wherein R¹, R^{1x}, R², R^{2x}, R^{x} and R^{y} are optionally substituted with one to four substituents selected each independently selected from alkyl, alkenyl, alkynyl, halo, hydroxyl, pseudohalo, amino, nitro, cycloalkyl, heterocyclyl, aryl and heteroaryl.

3. The compound of claim 2, wherein R¹ and R^{1x} are each independently —H, optionally substituted —O(C₁-C₂₄)alkyl; wherein at least one of R¹ and R^{1x} is not —H;
R² and R^{2x} are each independently —H, optionally substituted —O(C₁-C₇)alkyl;
R_{q} is a pharmacologically active phosphonate or a phosphonate derivative of a pharmacologically active compound of formula: 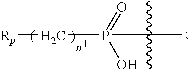
Rₚ is a pharmacologically active nucleoside or analog thereof; and
n¹ is 0 to 3.

4. The compound of claim 1, wherein Rₚ is 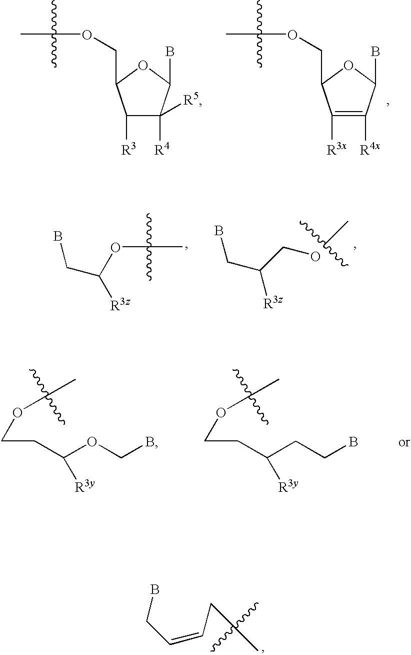
wherein
R³, R⁴ and R⁵ are each independently H, hydroxy, halo, azido, substituted or unsubstituted C₁₋₆ alkyl, substituted or unsubstituted C₂₋₆ alkenyl or substituted or unsubstituted C₂₋₆ alkynyl;
and wherein the substituents on the alkyl and alkenyl groups, when present, are selected from one to four alkyl, alkenyl, alkynyl, halo, hydroxyl, pseudohalo, amino, nitro, cycloalkyl, heterocyclyl, aryl or heteroaryl.
B is a purine or pyrimidine base or analog thereof;
R^{3x} is H, azido, substituted or unsubstituted C₁₋₆ alkyl, substituted or unsubstituted C₂₋₆ alkenyl or substituted or unsubstituted C₂₋₆ alkynyl;
R^{4x} is H, C₁₋₆ substituted or unsubstituted alkyl, C₂₋₆ substituted or unsubstituted alkenyl or C₂₋₆ substituted or unsubstituted alkynyl;
R^{3z} is H, substituted or unsubstituted C₁₋₆ alkyl, hydroxylC₁₋₆ alkyl, haloC₁₋₆ alkyl, azidoC₁₋₆ alkyl or OH; and
wherein the alkyl, alkenyl and alkynyl groups when substituted, are substituted with one to four substituents each independently selected from alkyl, alkenyl, alkynyl, halo, hydroxyl, pseudohalo, amino, nitro, cycloalkyl, heterocyclyl, aryl and heteroaryl.

5. The compound of claim 1, wherein R_{L} has formula:

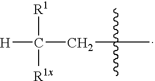

6. The compound of claim 1, wherein R_{L} has formula:

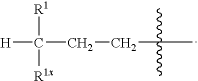

7. The compound of claim 1, wherein R_{L} has formula:

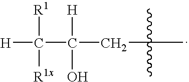

8. The compound of claim 1, wherein R_{L} is hexadecyloxypropyl, octadecyloxypropyl, or octadecyloxyethyl.

9. The compound of claim 4, wherein B is 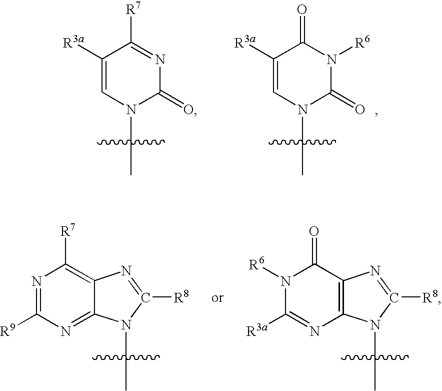
wherein R^{3a} is H, C₁₋₆ alkyl, C₂₋₆ alkenyl, C₂₋₆ alkynyl, C₃₋₆ cycloalkyl, hydroxy, halo, aryl or heteroaryl;
R⁶ is H or C₁₋₆ alkyl, C₂₋₆ alkenyl, C₂₋₆ alkynyl or cycloalkyl;
R⁷ is H, hydroxy, halo, C₁₋₆ alkyl, C₂₋₆ alkenyl, C₂₋₆ alkynyl, cycloalkyl or NR⁴R⁵;
R⁸ is H, C₁₋₆ alkyl, C₂₋₆ alkenyl, C₂₋₆ alkynyl or cycloalkyl and
R⁹ is H, C₁₋₆ alkyl, C₂₋₆ alkenyl, C₂₋₆ alkynyl, cycloalkyl, halo or NR⁴R⁵, where R⁴ and R⁵ are each independently H, C₁₋₆ alkyl, C₂₋₆ alkenyl, C₂₋₆ alkynyl, or C₃₋₆ cycloalkyl.

10. The compound of claim 9, wherein B is selected from:

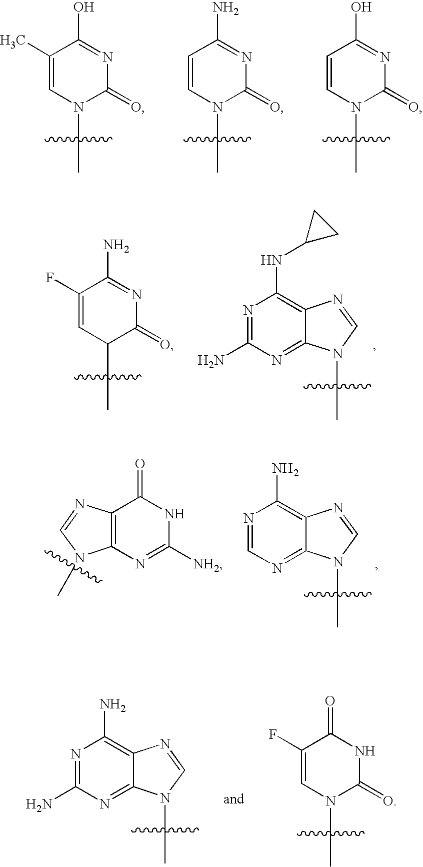

11. The compound according to claim 1, wherein R_{q} is an acyclic nucleoside phosphonate.

12. The compound of claim 11, wherein the acyclic nucleoside phosphonate is cidofovir.

13. The compound of claim 11, wherein the acyclic nucleoside phosphonate is (S)-HPMPA.

14. The compound of claim 11, wherein the acyclic nucleoside phosphonate is adefovir.

15. The compound of claim 11, wherein the acyclic nucleoside phosphonate is tenofovir.

16. The compound of claim 11, wherein the acyclic nucleoside phosphonate is PMEG.

17. The compound of claim 1, wherein R_{q} is a nucleoside-5′-phosphonate or a nucleoside-5′-methylene phosphonate.

18. The compound of claim 17, wherein the 5′-methylene phosphonate is azidothymidine.

19. The compound of claim 18, wherein the 5′-methylene phosphonate is 2′-O-methyl cytosine.

20. The compound of claim 18, wherein the 5′-methylene phosphonate is a β-D-1′-methyl ribofurano analog of cytidine, guanosine, uridine, adenosine, inosine or thymidine.

21. The compound of claim 18, wherein the 5′-methylene phosphonate is a β-D-2′-C-methyl ribofurano analog of cytidine, guanosine, uridine, adenosine, inosine or thymidine.

22. The compound of claim 18, wherein the 5′-methylene phosphonate is a β-D-2′-O-methyl ribofurano analog of cytidine, guanosine, uridine, adenosine, inosine or thymidine.

23. The compound of claim 1, wherein the compound has formula:

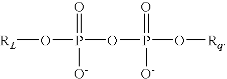

24. The compound of claim 1, wherein the compound has formula:

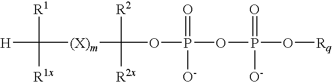

25. The compound according to claim 23, wherein R_{q} is an acyclic nucleoside phosphonate.

26. The compound of claim 25, wherein the acyclic nucleoside phosphonate is cidofovir.

27. The compound of claim 25, wherein the acyclic nucleoside phosphonate is (S)-HPMPA.

28. The compound of claim 25, wherein the acyclic nucleoside phosphonate is adefovir.

29. The compound of claim 25, wherein the acyclic nucleoside phosphonate is tenofovir.

30. The compound of claim 26, wherein the acyclic nucleoside phosphonate is PMEG.

31. The compound of claim 1 selected from HDP-phospho-(S)-HPMPA, ODE-phospho-(S)-HPMPA, OLE-phospho-(S)-HPMPA, OLP-phospho-(S)-HPMPA, 15-methyl-hexadecyloxy-propyl-phospho-(S)-HPMPA, 17-methyl-octadecyloxy-ethyl-phospho-(S)-HPMPA, 16-fluoro-hexadecyloxy-propyl-phospho-(S)-HPMPA, 18-fluoro-octadecyloxy-ethyl-phospho-(S)-HPMPA, 15-methyl-hexadecyloxy-ethyl-phospho-(S)-HPMPA, HDP-phospho-cidofovir, ODE-phospho-cidofovir, OLE-phospho-cidofovir, OLP-phospho-cidofovir, HDP-phospho-PMEG, ODE-phospho-PMEG, HDP-phospho-PME-DAP, HDP-phospho-PME-N⁶cPr-DAP, OLE-phospho-PME-N⁶cPr-DAP, HDP-phospho-PPMG, HDP-phospho-PPM-DAP, HDP-phospho-PPM-N⁶cPr-DAP, HDP-phospho-PME-5FU, HDP-phospho-PME-5FC, HDP-phospho-HPMP-5FC, HDP-phospho-HPMP-5FU, HDP-phospho-Phosphonomethoxy-3TC, HDP-phospho-Phosphonomethoxy-2′-C-methyl ribo-guanine, HDP-phospho-Phosphonomethoxy-1′-methyl cytidine, HDP-phospho-PM-2′-O-methyl cytidine and ODE-phospho-PM-2′-C-methyl adenosine.

32. The compound of claim 1 selected from HDP-phospho-(S)-HPMPA), ODE-phospho-(S)-HPMPA, OLE-phospho-(S)-HPMPA, OLP-phospho-(S)-HPMPA, 15-methyl-hexadecyloxy-propyl-phospho-(S)-HPMPA, and 17-methyl-octadecyloxy-ethyl-phospho-(S)-HPMPA.

33. A pharmaceutical composition comprising a compound of claim 1 and a pharmaceutically acceptable carrier.

34. A method for treating a viral infection, wherein the method comprises administering an effective amount of a compound of claim 1.

35. The method of claim 34, wherein the viral infection is a caused by influenza, hepatitis B virus, hepatitis C virus, cytomegalovirus, Varicella zoster virus, Herpes simplex virus types 1 and 2, Epstein-Barr virus, Herpes type 6 and type 8, Varicella zoster virus, Epstein Barr virus infections, retroviral infections, orthopox viruses, vaccinia, ebola virus, adenovirus or papilloma virus.

36. The method of claim 35, wherein the viral infection is Hepatitis B.

37. A method for treating a growing neoplasm, wherein the method comprises administering an effective amount of a compound of claim 1.

38. A method for modulating cell proliferation, wherein the method comprises administering an effective amount of a compound of claim 1.

39. A method for treating a cancer, wherein the method comprises administering an effective amount of a compound of claim 1.

40. The method of claim 39, wherein the cancer is selected from lung cancer, head and neck squamous cancers, colorectal cancer, prostate cancer, breast cancer, acute lymphocytic leukemia, adult acute myeloid leukemia, adult non Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, and skin cancer.

41. An article of manufacture, comprising packaging material and a compound of claim 1, contained within the packaging material, wherein the compound is effective for treatment of a disease associated with a viral infection or cell proliferation and the packaging material includes a label that indicates that the compound is used for treatment, prevention or amelioration of a disease associated with a viral infection or cell proliferation.

42-45. (canceled)
